(19) United States
(12) Patent Application Publication
(10) Pub. No.: US 2022/0249476 A1
(43) Pub. Date: Aug. 11, 2022

(54) MutL LOSS PREDICTS SENSITIVITY TO CDK4/6 INHIBITORS IN CANCER

(71) Applicant: Baylor College of Medicine, Houston, TX (US)

(72) Inventors: Svasti Haricharan, Houston, TX (US); Matthew J. Ellis, Houston, TX (US); Meenakshi Anurag, Cypress, TX (US); Matthew Bainbridge, Houston, TX (US)

(21) Appl. No.: 17/053,708

(22) PCT Filed: May 8, 2019
(86) PCT No.: PCT/US2019/031367

§ 371 Date: Nov. 6, 2020

Related U.S. Application Data
(60) Provisional application No. 62/670,368, filed on May 11, 2018.

(51) Int. Cl. A61K 31/506; A61K 31/519; A61K 45/06; C12Q 1/6886

(52) U.S. Cl. CPC A61K 31/506; C12Q 1/6886; A61K 45/06; A61K 31/519

(57) ABSTRACT

Embodiments of the disclosure concern determination of suitability of a particular type of therapy for ER+ cancers based on the presence of loss of members of double-strand break repair (such as MutL) and members of single-strand break repair. In particular, the loss of expression and/or mutation of CETN2, ERCC1, NEIL2, MLH1, and PMS2 is indicative of the particular ER+ cancer as being sensitive to one or more CDK-4/6 inhibitors. In specific cases, determination of loss of at least MutL renders the individual suitable for therapy with one or more CDK-4/6 inhibitors.

This application claims priority to U.S. Provisional Patent Application Ser. No. 62/670,368, filed May 11, 2018, which is incorporated by reference herein in its entirety.

### TECHNICAL FIELD

Embodiments of the disclosure include at least the fields of cell biology, molecular biology, diagnosis, prognosis, and medicine, including cancer medicine.

### BACKGROUND

Breast cancer is the most frequent form of cancer affecting women, and estrogen-receptor positive (ER+) tumors account for 60-70% of all reported cases. For patients with early stage ER+ disease, endocrine therapy: tamoxifen or an aromatase inhibitor (AI) are preferred first-line therapies. Despite these treatments, at least 1 in 4 patients develop fatal endocrine therapy resistance (1, 2). Although some markers predictive of endocrine treatment (ET) response are available, for example ERBB2 mutation/amplification (3, 4), gene expression profiles (5) and on-neoadjuvant endocrine treatment Ki67 analysis (6, 7), resistance largely remains an unpredictable and poorly understood event. Efforts to study underlying mechanisms of ET resistance have focused on activation of peptide growth factors (e.g. EGFR, ERBB2) and on activating mutations or translocation in ESR1 (8, 9). However, these mechanisms mostly explain adaptive or acquired resistance to endocrine treatment in the advanced setting, thereby circumscribing their use as predictive markers in primary tumors. The success of cyclin dependent kinase (CDK) 4/6 inhibition for metastatic breast cancer (10, 11) indicates a major role for the target cell cycle dependent kinases in restoring growth control in ET resistant tumors, but these agents lack accurate preductive markers. Consequently adjuvant treatment will lead to over-treatment in some patients and under-treatment in others.

It is known that loss of the MutL components of the mismatch repair (MMR) complex causes poor initial response to ET (intrinsic ET resistance) but in the MutL deficient setting tumors remain sensitive to CDK4/6 inhibition (12). Effects of other DNA damage response/repair (DDR) defects on ET resistance are understudied but might have similar relationships. In other cancer types, disruptions in DDR pathways associate with tumor formation, responsiveness to chemotherapy, and loss of replicative checkpoints in many cancer types(13). Additionally, ER-induced signaling and proliferation downregulates many DDR pathways in the normal mammary gland(14), potentially signifying a relationship between defects in DDR and hormonal response in ER+ breast cancer cells. Several genomic studies on breast cancer have identified signatures of DNA repair defects generated by classifying types of mutations, but the impact of these studies have been diluted by uncertainty regarding the molecular origin and clinical relevance of these signatures (15). Therefore, there is strong rationale for conducting a comprehensive, molecular analysis of the role for DDR defects in regulating and predicting ET response.

DDR is constituted of 8 canonical pathways: mismatch repair (MMR)—which can be further broken down into MutL and MutS complementation groups—nucleotide excision repair (NER), base excision repair (BER), non-homologous end joining (NHEJ), homologous recombination (HR), Fanconi Anemia (FA), trans-lesion synthesis (TLS) and direct repair (DR)(16). The first five of these pathways fall into one of two larger categories: single strand break repair (SSBR) consisting of MMR, BER and NER, and double strand break repair (DSBR) comprised of NHEJ and HR(17) (FIG. 1A).

The present disclosure includes embodiments that provide solutions to long-felt needs in the art to provide improved predictability of outcome with certain types of cancer, including ER+ cancer.

### BRIEF SUMMARY

Embodiments of the disclosure address the role of DNA damage repair (DDR) defects in poor outcome ER+ disease and associated methods and compositions. In particular embodiments, the disclosure concerns treatment protocols for individuals having susceptibility to particular therapies when the individual has a particular gene expression profile and/or certain mutations in one or more genes. In specific cases, an individual with ER+ cancer and having particular gene regulation dysfunctions allows the individual to be susceptible for treatment with one or more CDK-4/6 inhibitors. In particular cases, dysregulation of gene expression for CETN2, ERCC1, NEIL2, MLH1, and PMS2 and optionally PMS1 and mutations or copy number loss of Protein Kinase, DNA-Activated, Catalytic Polypeptide (PRKDC) allows the individual to be responsive to one or more CDK-4/6 inhibitors. The cancer may be ER+ breast, bladder, colorectal, lung, ovarian, prostate, stomach and/or hepatic cancer, and so forth.

In one embodiment, there is a method of determining efficacy of one or more cyclin D-dependent kinase (CDK) 4/6 inhibitors as therapy for an individual with ER+ cancer (including ER+ breast, bladder, colorectal, lung, ovarian, prostate, stomach or hepatic cancer), comprising the step of determining in a sample from the individual the level of expression and/or presence of mutation in Centrin 2 (CETN2), excision repair cross-complementation group 1 (ERCC1), Nei Like DNA Glycosylase 2 (NEIL2), mutL homolog 1 (MLH1), and PMS1 homolog 2, mismatch repair system component (PMS2). In at least some cases, when the expression level of CETN2, ERCC1, NEIL2, MLH1, and PMS2 in the sample from the individual is reduced compared to a standard or compared to normal levels and/or when there is a mutation in CETN2, ERCC1, NEIL2, MLH1, and PMS2, the individual is provided one or more CDK-4/6 inhibitors. The method may further comprise the step of determining the level of expression and/or presence of mutation of PMS 1 homolog 1, mismatch repair system component (PMS1) in the sample and/or determining copy number loss or mutation of Protein Kinase, DNA-Activated, Catalytic Polypeptide (PRKDC), for example. The one or more CDK-4/6 inhibitors may comprise abemaciclib, palbociclib, ribociclib, or a combination thereof, for example. The individual may also be provided hormone therapy, surgery, chemotherapy, or radiation for the cancer. The sample may be of any kind, including biopsy, urine, feces, polyp, cerebrospinal fluid, blood, semen, nipple aspirate, or a combination thereof, for example.

In a certain embodiment, there is a method of treating an individual for ER+ cancer, comprising the step of providing to an individual with reduced expression levels of CETN2, ERCC1, NEIL2, MLH1, and PMS2 compared to a standard or compared to normal levels, and/or has one or more mutations in CETN2, ERCC1, NEIL2, MLH1, and PMS2, an effective amount of one or more CDK-4/6 inhibitors.

In one embodiment, there is a method of treating an individual for ER+ cancer, comprising the steps of (a) identifying a subject susceptible to treatment of ER+ cancer by: (1) measuring from a sample (biopsy, urine, feces, polyp, cerebrospinal fluid, blood, semen, nipple aspirate, or a combination thereof, for example) from the individual the level of expression of CETN2, ERCC1, NEIL2, MLH1, and PMS2 compared to a standard or compared to normal levels; and/or (2) assaying from a sample from the individual for one or more mutations in the gene, mRNA, or protein of CETN2, ERCC1, NEIL2, MLH1, and PMS2; and (b) administering an effective amount of one or more CDK-4/6 inhibitors to the individual that is susceptible to treatment of ER+ cancer by having reduced expression levels of CETN2, ERCC1, NEIL2, MLH1, and PMS2 and/or by having one or more mutations in each of CETN2, ERCC1, NEIL2, MLH1, and PMS2.

In particular embodiments, an individual is provided one or more therapies for the ER+ cancer (including breast, bladder, colorectal, lung, ovarian, prostate, stomach or hepatic cancer) other than the one or more CDK-4/6 inhibitors (abemaciclib, palbociclib, ribociclib, or a combination thereof, for example), including hormone therapy, surgery, radiation, chemotherapy, or a combination thereof. The hormone therapy may comprise one or more selective estrogen-receptor response modulators (SERMs); one or more Aromatase inhibitors; one or more Estrogen-receptor downregulators (ERDs); one or more Luteinizing hormone-releasing hormone agents (LHRHs); or a combination thereof. In specific embodiments, the method further comprises the step of measuring the level of expression and/or assaying for mutation in PMS1 and/or determining copy number loss or mutation of PRKDC.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1B. General embodiments of the disclosure. FIG. 1A) Network view of different DDR pathways along with the shared genes (gray nodes) and unique genes (indicated by name, adjacent to pathway name). Pathways associated with SSBR are denoted as yellow nodes and DSBR denoted as orange nodes. Lines indicate pathways that share common genes. Mismatch repair (MMR), nucleotide excision repair (NER), base excision repair (BER), non-homologous end joining (NHEJ), homologous recombination (HR), Fanconi Anemia (FA), trans-lesion synthesis (TLS) and direct repair (DR). FIG. 1B) Schematic representation of design of screening approach to identify DDR pathways and genes associated with ET response. Grey boxes indicate data accrued at baseline and cyan boxes indicate data accrued on-AI treatment. Supporting data with detailed schema is presented in FIG. 7.

FIGS. 2A-2D. RNA levels of MMR, BER and NER genes associate inversely with on-endocrine treatment Ki67. FIG. 2A) Table describing 13 candidate genes with significant correlation between RNA levels and on-treatment Ki67 in ER+ tumors from NeoAI. Pearson correlation analysis was used to determine the correlation coefficient. False discovery rate (FDR) is denoted in the table for specific correlations. Blue boxes indicate correlations where FDR<20%. Pathways to which candidate genes belong are noted in the DDR function column. Yellow boxes indicate SSBR pathways and orange boxes, DSBR. As a positive control for the analysis, three genes previously implicated in response to ET: ESR1, GATA3 and RUNX1, are included. FIG. 2B) Bar graph indicating enrichment for SSBR genes in the list of 13 candidate genes. Fisher's exact test determined p-value. FIG. 2C) Venn diagram depicting proportion of genes from each DDR pathway that was implicated in ET resistance from correlation analysis in A. Blue circle indicates candidate gene population. FIG. 2D) KEGG pathway enrichment analysis of the candidate gene list against all DDR genes used in the analysis revealed significant enrichment of indicated pathways. Number of genes from candidate list contributing to each enriched pathway is listed along the bars. A −log10 (p-value) of 1.2 denotes a p<0.05.

FIGS. 3A-3E. CETN2, NEIL2 and ERCC1 loss associates with poor survival of ER+ breast cancer patients. FIG. 3A) Forest plot summarizing results of multivariate analysis of the 13 candidate genes in METABRIC. Other factors included in the analysis were tumor size, grade and node positivity. Boxes denote hazard ratio (HR) based on overall survival outcome, and error bars the 95% confidence interval. HR for genes whose dysregulation associated with poor survival (p≤0.05) by univariate analysis (presented in FIG. 8) are shown as red boxes. FIG. 3B-FIG. 3D) Kaplan-Meier curves depicting disease specific survival of patients with luminal breast cancer treated with ET whose tumors have low (mean-1.5 standard deviation) CETN2 (3B), NEIL2 (3C) and ERCC1 (FIG. 3D) expression (red) in METABRIC data set. Kaplan-Meier curves for HER2-enriched and basal-like tumors are presented in FIG. 9. (FIG. 3E) Kaplan-Meier curves depicting recurrence free survival of tamoxifen treated ER+ breast cancer patients whose tumors had low expression of CETN2, ERCC1 and NEIL2 (CEN Low in red) in Loi data set. Individual Kaplan-Meier curves presented in FIG. 10. All HRs were calculated using Cox Regression and log-rank p-value determined significance of differences in survival.

FIGS. 4A-4D. NER, BER and NHEJ genes are enriched for damaging mutations in endocrine treatment resistant tumors. FIG. 4A) Enrichment analysis for prevalence of predicted damaging mutations (based on SIFT scores: lower the SIFT score, the more damaging the mutation is predicted to be) in SSBR and DSBR pathways compared to genome-wide prevalence in tumors from NeoAI. Significant p-values were determined by Wilcoxon test analysis. Similar analysis for each individual DDR pathway is presented in FIG. 11. FIG. 4B) Pie charts comparing proportion of missense (light yellow—SSBR, light orange—DSBR) and frameshift/nonsense (yellow—SSBR, orange—DSBR) mutations in SSBR and DSBR genes relative to proportion in control gene set (grey). Z-statistic for two population proportions was used to determine significant differences in proportion of mis sense to frameshift/nonsense mutations in patients who remained alive to maintain adequate sample size for the test. FIG. 4C) Forest plots depicting hazard ratios for overall survival of patients from TCGA (above) and MSKCC-IMPACT (below) with ER+ tumors harboring non-synonymous mutations in indicated pathways. Log rank test was used to determine significance and Cox Regression Proportional Hazards generated univariate hazard ratios. Supporting data investigating a role for NHEJ gene mutation in ER+ breast cancer survival is presented in FIG. 12, and analyses controlling for replication defects, genome instability and mutation load are presented in FIGS. 13-14. FIG. 4D) Venn diagram and word cloud (yellow text, SSBR and orange text, DSBR) summarizing candidate pathways that significantly associate with poor survival of ER+ breast cancer patients (red) based on mutational (green) or transcriptomic (violet) dysregulation. MMR, NER and BER pathways are identified at the intersection of all analyses. Larger font size indicates greater confidence.

FIGS. 5A-5F. Inhibition of CETN2, NEIL2 and ERCC1 induces resistance to all classes of endocrine therapy in ER+ breast cancer cells and PDXs. (FIG. 5A) Western blot validation of siRNA-mediated knockdown of CETN2, NEIL2 and ERCC1 respectively in MCF7 cells. Results from three independent experiments are depicted. Columns represent the mean and error bars the standard deviation. RNA level validation of knockdown is presented in FIG. 15A. (FIG. 5B-FIG. 5D) Dose response curves of MCF7 cells with transient inhibition of CETN2, NEIL2 or ERCC1 treated with fulvestrant (FIG. 5B) or 4-hydroxy-tamoxifen (FIG. 5C). Dose response to estrogen stimulation is presented in FIG. 15B. IC50 values were calculated from three independent dose curves for each condition and Student's t-test used to determine significant differences in IC50 values. nM, nanomolar. Independent validation in a second cell line was determined, and orthogonal validation of knockdown results are presented in FIG. 16. (FIG. 5D) Box plot depicting tumor viability in vivo after anastrozole treatment of 7 ER+ PDX lines from BCaPE, calculated using area under the curve (AUC) measurements. CEN: CETN2, ERCC1, NEIL2; MP: MLH1, PMS2. Wilcoxon Rank Sum test determined p-value. (FIG. 5E) Working model indicating peak expression levels of NEIL2, ERCC1, MLH1, and CETN2 genes across the cell cycle. Data generated from two independent double thymidine block experiments (www.dnarepairgenes.com). Cumulative peak expression level of all genes in NHEJ, HR and FA pathways also indicated. Y-axis indicates relative gene expression level and X-axis is plotted based on number of hours post release of double thymidine block. Implication of CDKs and estrogen stimulation in the cell cycle is based on published reports. Supporting mechanistic data is presented in FIGS. 17-18. (FIG. 5F) Bar graphs represent growth inhibition, relative to vehicle treated cells, in response to 100 nM of fulvestrant or 1 μM of Palbociclib, CDK4/6 inhibitor in MCF7 cells stably expressing pooled RNAi oligos against CETN2, ERCC1, NEIL2 or scrambled control. Student's t-test determined p-values by comparing growth inhibition in response to Palbociclib against that in response to fulvestrant.

FIGS. 6A-6E. Cumulative incidence and predictive potential of CETN2, NEIL2, ERCC1, MLH1 and PMS2 (CENMP) deficiency. FIG. 6A-FIG. 6B) Stacked columns indicating cumulative frequency of dysregulation (mutation or underexpression) of CETN2, ERCC1, NEIL2 (CEN-); MLH1 , PMS2 (MutL-); and PRKDC mutation (mut) or copy number loss (cnl) in ER+ breast tumors from METABRIC (FIG. 6A) and TCGA (FIG. 6B). Fisher's exact test determined p-values. FIG. 6C) Box plots describing CENMP expression signature score in tumors from patients based on their response to AI-treatment. Wilcoxon Rank Sum test determined p-values. 6D-6E) Kaplan-Meier survival curves evaluating separation based on CENMP score in ET treated ER+ patients from METABRIC (FIG. 6D) and Loi (FIG. 6E) data sets. Cox Regression identified hazard ratio (HR) and log rank test determined p-values for survival analyses.

FIGS. 7A-7B. Set-up of transcriptional analysis. FIG. 7A) Correlation between on-treatment Ki67 (IHC) and MKi67 (mRNA) across Z1031/POL cohort. Regression analysis identified p-value and Pearson's correlation coefficient. FIG. 7B) Work flow for transcriptomic analysis. Schematic representation of correlation analysis of baseline DDR Gene expression (mRNA) and on-treatment Ki67 (mRNA and IHC) and subsequent validation of candidates in METABRIC. ET, endocrine treatment.

FIG. 8. Low CETN2, NEIL2 and ERCC1 RNA levels associate with worse overall survival. Forest plot summarizing results of univariate analysis of the thirteen candidate genes in METABRIC. Boxes denote hazard ratio (HR) based on overall survival outcome calculated using Cox Regression analysis of Proportional Hazards. HR for genes whose dysregulation associated with poor survival (p<=0.05) are shown as red boxes. Error bars indicate 95% confidence intervals.

FIGS. 9A-9F. RNA levels of CETN2, NEIL2 and ERCC1 do not associate with survival in basal-like or HER2 enriched tumors. Kaplan-Meier curves for disease specific survival of patients from METABRIC with HER2-enriched (FIG. 9A-FIG. 9C) and basal-like (FIG. 9D-FIG. 9F) breast cancer whose tumors have low RNA levels of CETN2, ERCC1 and NEIL2. The low expressing tumor sets have been shown in red. Log rank test was used to determine significance and Cox Regression Proportional Hazards generated univariate hazard ratios (HR).

FIGS. 10A-10C. Low RNA levels of CETN2, NEIL2 and ERCC1 correlate with worse recurrence-free survival in ER+ breast cancer patients. Kaplan-Meier curves depicting recurrence-free survival of luminal breast cancer patients who were treated with tamoxifen and whose tumors harbored low CETN2 (FIG. 10A), NEIL2 (FIG. 10B) and ERCC1 (FIG. 10C) expression from Loi dataset. Log rank test was used to determine significance and Cox Regression Proportional Hazards generated univariate hazard ratios (HR).

FIG. 11. NER, BER, HR and NHEJ genes are enriched for damaging mutations in ER+ patient tumors. Histogram depicting inverse log10 values of FDR of over-represented KEGG pathways in genesets which harbor damaging (red) or tolerant mutations when compared against all protein coding genes in humans using Webgestalt.

FIGS. 12A-12D. Loss of PRKDC associates with poor survival in ER+ breast cancer patients. FIG. 12A-FIG. 12B, FIG. 12D) Kaplan-Meier survival curves describing patients whose ER+ tumors harbor mutations (mut) or copy number loss (cnl) in all genes from non-homologous end joining (NHEJ) pathways (FIG. 12A), or in PRKDC vs other NHEJ genes (FIG. 12B, FIG. 12D) from TCGA (FIG. 12A, FIG. 12B) and METABRIC (FIG. 12D) datasets. Cox Regression analysis identified univariate hazard ratios (HR) and log-rank test determined p-values. FIG. 12C) Box plot describing PRKDC RNA levels in ER+ tumors with either copy number loss (Loss) of, or mutations in, PRKDC. Wilcoxon Rank Sum test determined p-value.

FIG. 13. Expression of replication genes does not significantly inversely correlate with on-treatment Ki67 levels in ER+ patient tumors. Correlation plots between replication genes—RPA1,2,3,4 and on-treatment Ki67 (On-tx Ki67) along with on-treatment Mki67 (On-tx Mki67 mRNA) in NeoAI dataset. Adjusted R reflects Pearson's correlation coefficient.

FIGS. 14A-14D. ER+ tumors with mutations in SSBR genes associate with higher mutation load. Box plot representing comparison of genomic instability index (FIG. 14A-FIG. 14B) and mutation load (FIG. 14C-FIG. 14D) in tumors with frameshift/nonsense (FS/NS) or missense (MS) mutations in SSBR (FIG. 14A, FIG. 14C) and DSBR (FIG. 14B, FIG. 14D) genes respectively when compared against rest of the tumors. Wilcoxon Rank Sum test determined p-values.

FIGS. 15A-15F. Inhibition of CETN2, NEIL2 and ERCC1 expression in ER+ breast cancer cells induces endocrine treatment resistant growth. FIG. 15A & FIG. 15C) qRT-PCR validation of transient siRNA and stably selected RNAi-mediated knockdown of CETN2, NEIL2 and ERCC1 in MCF7 (FIG. 15A) and ZR75 (FIG. 15C) cells. Results from six independent experiments are depicted. Columns represent the mean and error bars the standard deviation. GOI, Gene Of Interest. FIG. 15B, FIG. 15D-FIG. 15F) Dose response curves of MCF7 (FIG. 15B) and ZR75 (FIG. 15D-FIG. 15F) cells with transient inhibition of CETN2, NEIL2 or ERCC1 treated with estradiol in charcoal-stripped serum (FIG. 15B & FIG. 15D), fulvestrant (15E) or 4-hydroxy-tamoxifen (FIG. 15F). Student's t-test comparing IC50 values from three independent replicates for each si-treatment against siScr determined p-values.

FIGS. 16A-16F. Inhibition of CETN2, NEIL2 and ERCC1 expression in ER+ breast cancer cells is causal to endocrine treatment resistant growth. FIG. 16A) qRT-PCR validation of transient siRNA-mediated knockdown of POLM and RAD23B respectively in MCF7 cells. Results from three independent experiments are depicted. Columns represent the mean and error bars the standard deviation. FIG. 16B-FIG. 16C, FIG. 16E-FIG. 16F) Dose response curves of MCF7 cells with transient inhibition of POLM and RAD23B (FIG. 16B-FIG. 16C), or with stably selected RNAi-mediated knockdown of CETN2, NEIL2 or ERCC1 treated with fulvestrant (FIG. 16B & FIG. 16E) and 4-hydroxy-tamoxifen (FIG. 16C & FIG. 16F). Student's t-test determined p-values by comparing IC50 values from three independent replicates for each si-treatment against siScr. FIG. 16D) Western blot validation of knockdown of CETN2, ERCC1 and NEIL2 at the protein level after stable selection for their respective RNAi in MCF7 cells. Quantification based on 4 independent experiments. β-actin serves as loading control.

FIGS. 17A-17E. Dysregulation of CETN2, NEIL2 and ERCC1 expression in ER+ breast cancer cells alters regulation of G1/S transition of the cell cycle. FIG. 17A & FIG. 17D) Histograms representing percentage of siScr, siCETN2, siNEIL2, siERCC1 and siMLH1 MCF7 cells that are Ki67 (FIG. 17A) and PCNA (FIG. 17D) positive after 48 hours of treatment with 100 nM of fulvestrant. Columns represent the mean and error bars the standard deviation. Student's t-test determined p-values. FIG. 17B-FIG. 17C) Boxplots indicating levels of CDK4 mRNA and PCNA protein level in CETN2, ERCC1, NEIL2, PRKDC proficient (CEN+PRKDCwt) and deficient (CEN-PRKDCmut) ER+ breast tumors based on values from microarray and RPPA data from TCGA. Wilcoxon Rank Sum test determined p-values. FIG. 17E) Histograms depicting percent growth inhibition in fulvestrant and abemaciclib (CDK4/6i)-treated MCF7 cells stably selected for RNAi-mediated knockdown of control, CETN2, ERCC1 or NEIL2, relative to vehicle treated cells. Columns represent the mean and error bars the standard deviation. Student's t-test determined p-values.

FIG. 18. ESR1-mediated regulation is not common to CETN2, NEIL2 and ERCC1 in ER+ breast cancer cells. Regression plots demonstrating direct correlation between ESR1 and PGR expression with that of CETN2, but not with that of NEIL2 or ERCC1 in ER+ patient tumors from NeoAI. R represent the Pearson's Correlation Coefficient.

FIGS. 19A-19B. MutL loss in bladder and colorectal cancer. MutL loss through mutation or low RNA levels associates with worse survival in CRC (FIG. 19A) and in the luminal subset of bladder cancer (FIG. 19B). Tumors with dysregulation of both MutL and MutS pathways (accounting for almost half the MutL-dysregulated tumors in CRC, and about 15% of bladder cancer samples) were left out of this comparison

FIGS. 20A-20B. Response of MutL⁻ cancer cells to CDK4/6 inhibition. Dose response to abemaciclib, a CDK4/6 inhibitor, in colorectal (FIG. 20A) and bladder (FIG. 20B) cancer cells that are endogenously MutL⁻ (yellow), endogenously MutL⁺ (blue), or engineered to be MutL− with siRNA against MLH1 (blue outline).

FIG. 21. Inverse correlation between frequency of MutL dysregulation and response to CDK4/6 inhibitor, Palbociclib/

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Each embodiment described herein may be combined with any other embodiment described herein.

A “patient”, “subject”, or “individual” are used interchangeably and refer to either a human or a non-human animal These terms include mammals, such as humans, primates, livestock animals (e.g., bovines, porcines), companion animals (e.g., canines, felines) and rodents (e.g., mice and rats).

As used herein, “treating” ER+ cancer refers to taking steps to obtain beneficial or desired results, including clinical results. Beneficial or desired clinical results include, but are not limited to, alleviation or amelioration of one or more symptoms associated with diseases or conditions.

As used herein, “administering” or “administration of” a compound or an agent to a subject can be carried out using one of a variety of methods known to those skilled in the art. For example, a CDK-4/6 inhibitor compound can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation, or administered using a device for such slow or extended release.

The term “therapeutically effective amount” is used herein to mean an amount or dose sufficient to cause an improvement in at least one symptom of ER+ cancer.

### I. General Embodiments

Embodiments of the disclosure encompass treatment programs for individuals that have ER+ cancer and are in need of knowing whether or not they are susceptible or resistant to a particular therapy. In particular embodiments, the individual is determined as susceptible to the therapy when there is dysregulation of one or more DNA damage repair (DDR) pathway members, such as at least mismatch repair (MMR) pathway members.

The disclosure concerns any cancers, including at least ER+ breast, colorectal, bladder, lung, ovarian, prostate, stomach and hepatic cancer. Estrogen receptor positive (ER+) breast cancer is treatable with endocrine drugs that interrupt estrogen receptor function, but fatal drug resistance occurs in at least 30% of cases. A comprehensive analysis of the molecular landscape of DNA repair defects in ER+ breast cancer patient tumors provided herein identifies defects in select DNA repair pathways as a novel class of endocrine treatment resistance drivers occurring in ˜40% of endocrine treatment resistant ER+ breast cancer patients. Candidate DNA damage repair genes identified are experimentally shown to be linked by common upregulation at the G1/S transition, suggesting that loss of DNA proof reading pathways disrupts ER regulation of the cell cycle, and therefore, response to endocrine treatment. A combined DDR signature score was developed that predicted poor outcome in multiple patient cohorts, which will have immense translational implications in stratifying patients who would not respond to endocrine therapy but will be good candidates for CDK4/6 inhibitors based treatment. These findings, therefore, significantly increase understanding of factors underlying response to standard-of-care in ER+ breast cancer patients and identify an alternative targeted therapeutic strategy for this subset of patients.

In specific method steps (that are only exemplary), expression and mutational status of DDR genes in ER+ breast tumors were correlated with proliferative response in neoadjuvant aromatase inhibitor therapy trials (discovery data set), with outcomes in METABRIC, TCGA and Loi data sets (validation data sets), and in patient derived xenografts. A causal relationship between candidate DDR genes and endocrine treatment response, and the underlying mechanism, was then tested in ER+ breast cancer cell lines. Correlations between loss of expression of at least three genes: CETN2 (p<0.001) and ERCC1 (p=0.01) from the nucleotide excision repair (NER) and NEIL2 (p=0.04) from the base excision repair (BER) pathways were associated with endocrine treatment resistance in discovery data sets, and subsequently validated in independent patient cohorts. Complementary mutation analysis supported associations between mutations in NER and BER pathways and reduced endocrine treatment response. A causal role for CETN2, NEIL2 and ERCC1 loss in intrinsic endocrine resistance was experimentally validated in ER+ breast cancer cell lines, and in ER+ patient-derived xenograft models. Loss of CETN2, NEIL2 or ERCC1 induced endocrine treatment response by dysregulating G1/S transition, and therefore, increased sensitivity to CDK4/6 inhibitors. A combined DDR signature score was developed that predicted poor outcome in multiple patient cohorts. Thus, the disclosure identifies at least DDR defects as a new class of endocrine treatment resistance drivers and indicates new avenues for predicting efficacy of CDK4/6 inhibition in the adjuvant treatment setting.

The methods of this disclosure can be used together with any known diagnostic or prognostic methods, such as physical inspection, visual inspection, biopsy, scanning, histology, radiology, imaging, ultrasound, use of a commercial kit, genetic testing, immunological testing, analysis of bodily fluids, or monitoring activity.

### II. Measuring Gene Expression to Determine Susceptibility to Treatment

In embodiments of the disclosure, the gene expression of certain genes is determined from a sample from an individual with ER+ cancer to provide information for the individual related to treatment susceptibility, as opposed to resistance. A treatment is considered susceptible when the treatment results in improvement of at least one symptom of the ER+ cancer (such as reduction in tumor size, reduction in the number of tumors, delay in onset of additional tumors, prevention of metastasis, slowing the growth of one or more tumors, and so forth). In some cases, the gene expression analysis concerns DDR gene expression downregulation. In certain embodiments, measurement of expression of more than one gene is required to make the determination of treatment efficacy for the individual. In specific cases the genes include at least CETN2, ERCC1, NEIL2, MLH1, and PMS2, and optionally PMS1 and PRKDC. Particular embodiments provide for the analysis of expression levels for all of CETN2, ERCC1, NEIL2, MLH1, and PMS2 (and optionally PMS1, and mutation or copy number loss of PRKDC) to be able to determine sensitivity to certain treatments, such as one or more CDK-4/6 inhibitors. In alternative embodiments, the expression levels are determined for less than all of CETN2, ERCC1, NEIL2, MLH1, and PMS2, such as 1, 2, 3, or 4 of CETN2, ERCC1, NEIL2, MLH1, and PMS2.

Measuring of the gene expression levels of any of the genes may occur by any suitable method. Characterization of gene expression patterns may occur through quantification of messenger RNA (mRNA) by such procedures as Reverse transcription quantitative PCR; DNA microarray analysis; RNA sequencing (RNA-seq); or a combination thereof.

In certain cases, the nucleic acid level can be determined by any methods known in the art to detect genotypes, single nucleotide polymorphisms, gene mutations, gene copy numbers, DNA methylation states, DNA acetylation states, chromosome dosages, etc. Exemplary methods for gene expression measuring (or gene product analysis) include, but are not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophisis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser deorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, HpaII tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, co-amplification at lower denaturation temperature-PCR (COLD-PCR), multiplex PCR, methyl-binding PCR analysis, or a combination thereof.

In specific embodiments, measurement of the gene(s) utilizes targeting of the mRNA of the gene using sequence that specifically targets the particular mRNA. In doing so, at least part of the sequence of the mRNA must be known. An example of CETN2 nucleotide sequence is in NCBI's GenBank® database Accession No. NM_004344. An example of NEIL2 nucleotide sequence is in GenBank® database Accession No. NM_145043. An example of ERCC1 nucleotide sequence is in GenBank® database Accession No. NM_202001. An example of PRKDC nucleotide sequence is in GenBank® database Accession No. NM_006904. An example of MLH1 nucleotide sequence is in GenBank® database Accession No. NM_000249. An example of PMS2 nucleotide sequence is in GenBank® database Accession No. NM_000535. An example of PMS1 nucleotide sequence is in GenBank® database Accession No. M_000534.

In specific cases, a level can be, without limitation, a genotypic level, a single nucleotide polymorphism level, a gene mutation level, a gene copy number level, a DNA methylation level, a DNA acetylation level, a chromosome dosage level, a gene expression level, or a combination thereof.

The skilled artisan recognizes that one can enhance the accuracy of gene expression measurement by normalization to reference genes, RNA quantity, to a standard, or control, for example.

### III. Assaying for Mutation(s) to Determine Susceptibility to Treatment

As an alternative, or in addition to, measuring gene expression levels to ascertain susceptibility to treatment, one may assay for a mutation in the corresponding gene, mRNA, or expressed protein. In specific embodiments, a mutation in one or more of CETN2, ERCC1, NEIL2, MLH1, and PMS2 (and optionally PMS1 and PRKDC) that leads to dysfunctional gene product (mRNA and/or protein) would indicate that the individual is a candidate for CDK-4/6 treatment. Mutations leading to dysfunction mRNA, for example, includes mutations that result in a truncated mRNA and/or mutations in the gene that result in a mutated mRNA and protein having an amino acid substitution, deletion, inversion, truncation, and so forth. Mutations leading to dysfunctional proteins, for example, may disrupt the wild-type activity of the protein.

In some cases, dysregulation of expression levels of a subset of CETN2, ERCC1, NEIL2, MLH1, and PMS2 (and optionally PMS1) coupled with mutation in the other gene products in the combination would indicate that the individual would be susceptible to CDK-4/6 treatment.

Assaying for mutations in a protein may occur by any suitable means. In specific embodiments, analysis of proteins for mutation(s) include sequencing of the protein, western blot analysis (for example, to identify truncations), functional analysis for loss of activity, disruption of secondary structure of the protein, and/or disruption of protein-protein interactions. Antibodies of any kind to the respective proteins may be employed (includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F(ab′).sub.2, and Fv)). The assaying may require the sequence of the protein. An example of CETN2 protein is at GenBank® database Accession No. NP_004335.1. An example of NEIL2 protein is at NP_659480.1. An example of ERCC1 protein is at NM_202001. An example of PRKDC protein is at NP_008835. An example of MLH1 protein is at NP_000240. An example of PMS2 protein is at NP_000526. An example of PMS1 protein is at NP_000525.

In embodiments of the invention, a protein level may be determined, such as a protein expression level, a protein activation level, or a combination thereof. In some embodiments, a protein activation level can comprise determining a phosphorylation state, an ubiquitination state, a myristoylation state, or a conformational state of the protein.

A protein level can be detected by any methods known in the art for detecting protein expression levels, protein phosphorylation state, protein ubiquitination state, protein myristoylation state, or protein conformational state. In some embodiments, a protein level can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser deorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microscopy, microfluidic chip-based assays, surface plasmon resonance, sequencing, Western blotting assay, or a combination thereof.

### IV. Methods of Treating

Embodiments of the disclosure include methods of treating an individual for ER+ cancer (of any stage) by administering to the individual an effective amount of one or more CDK-4/6 inhibitors. The CDK-4/6 inhibitor(s) may be of any kind, including at least abemaciclib, palbociclib, ribociclib, or a combination thereof, merely as examples. The administering step may occur as a result of obtaining information from the suitability of the individual in question for the CDK-4/6 inhibitor treatment. For an example, the administering step may only occur when it has been determined that the individual is susceptible to one or more CDK-4/6 inhibitors based on having certain gene expression patterns and/or certain gene product dysregulation.

In particular embodiments, the method of treatment includes administering an effective amount of one or more CDK-4/6 inhibitors to the individual that is susceptible to treatment of ER+ cancer by having reduced expression levels of CETN2, ERCC1, NEIL2, MLH1, and PMS2 (and optionally PMS1 or copy number loss of PRKDC) and/or by having one or more mutations in each of (or a subset of) CETN2, ERCC1, NEIL2, MLH1, and PMS2 and PRKDC.

In certain embodiments, the individual may be resistant to standard-of-care therapy for ER+ cancer but the determination of the particular gene expression pattern and/or certain gene product dysregulation identifies that the individual will be sensitive to one or more CDK-4/6 inhibitors. In some cases, the individual is given surgery, hormone therapy, radiation, or other therapies in addition to the one or more CDK-4/6 inhibitors.

The individual may receive one or more doses of one or more CDK-4/6 inhibitors. Successive doses may or may not include the same one or more CDK-4/6 inhibitors. In some cases, the one or more CDK-4/6 inhibitors are administered to the individual daily, more than once daily, weekly, biweekly, monthly, and so forth.

In a clinical example of treatment, an individual may have been determined to have ER+ cancer, for example upon determination of the cancer cells having the estrogen receptor based on immunohistochemistry (IHC) analysis or adjudged to be luminal by PAM50 subtyping. The individual may or may not receive therapy before it is determined whether or not the individual would be susceptible to one or more CDK-4/6 inhibitors based on methods of the disclosure. For example, the individual may receive one or more standard-of care treatments (as an example, hormone therapy) and it is determined that the individual is or is not resistant to the standard-of-care treatment(s). The individual may then be tested for being susceptible to treatment with one or more CDK-4/6 inhibitors based on methods of the disclosure. Upon determination that the individual has the particular gene expression pattern and/or certain gene product dysregulation as described herein, the individual is then provided one or more CDK-4/6 inhibitors.

Embodiments of the disclosure encompass methods of treating an individual for ER+ cancer, comprising the step of providing to an individual with reduced expression levels of CETN2, ERCC1, NEIL2, MLH1, and PMS2 compared to a standard and/or compared to normal levels, and/or has one or more mutations in CETN2, ERCC1, NEIL2, MLH1, and PMS2, an effective amount of one or more CDK-4/6 inhibitors.

Embodiments of the disclosure include methods of treating an individual for ER+ cancer, comprising the steps of: (a) identifying a subject susceptible to treatment of ER+ cancer by: (1) measuring from a sample from the individual the level of expression of CETN2, ERCC1, NEIL2, MLH1, and PMS2 compared to a standard or compared to normal levels; and/or (2) assaying from a sample from the individual for one or more mutations in the gene, mRNA, or protein of CETN2, ERCC1, NEIL2, MLH1, and PMS2; and (b) administering an effective amount of one or more CDK-4/6 inhibitors to the individual that is susceptible to treatment of ER+ cancer by having reduced expression levels of CETN2, ERCC1, NEIL2, MLH1, and PMS2 and/or by having one or more mutations in each of CETN2, ERCC1, NEIL2, MLH1, and PMS2.

In some embodiments, there is a method of prognosing and/or diagnosing a patient comprising a) measuring the level of expression of one or more of CETN2, ERCC1, NEIL2, MLH1, and PMS2 in a sample from the patient; b) comparing the level of expression to a control sample(s) or control level(s) of expression; and, c) prognosing or diagnosing the patient based on the levels of measured expression. In specific embodiments, the one or more of CETN2, ERCC1, NEIL2, MLH1, and PMS2 are alternatively or additionally assayed for one or more mutations.

In any methods of the disclosure, the individual or patient may have been or may be determined to have a familial history of ER+ cancer, although in some cases the the individual or patient has been determined not to have a familial history of ER+ cancer. The individual or patient may or may not have had a prior screening procedure for ER+ cancer. The patient may or may not have been diagnosed with ER+ cancer. In specific aspects of the methods, the expression level of no other ER+ cancer biomarker in the biological sample was determined.

### V. Pharmaceutical Preparations

Pharmaceutical compositions of the present invention comprise an effective amount of one or more CDK-4/6 inhibitors dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one or more CDK-4/6 inhibitors or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The one or more CDK-4/6 inhibitors may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more CDK-4/6 inhibitors may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include one or more CDK-4/6 inhibitors, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more CDK-4/6 inhibitors may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a particular dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In some embodiments, the dose comprises 25 mg/day-1000 mg/day or more, including 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 125 mg/day, 150 mg/day, 200 mg/day, 225 mg/day, 250 mg/day, 300 mg/day, 350 mg/day, 400 mg/day, 450 mg/day, 500 mg/day, 750 mg/day, or 1000 mg/day or more.

### A. Alimentary Compositions and Formulations

In particular embodiments of the present disclosure, the one or more CDK-4/6 inhibitors are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft- shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosuremay alternatively be incorporated with one or more excipients in the form of a mouthwash, dentifrice, buccal tablet, oral spray, or sublingual orally- administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and particularly about 1% to about 2%.

### B. Parenteral Compositions and Formulations

In further embodiments, one or more CDK-4/6 inhibitors may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is considered to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, particular methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other particular embodiments of the disclosure, the active compound one or more CDK-4/6 inhibitors may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Kits of the Disclosure

Any of the compositions described herein may be comprised in a kit. In a non-limiting example, one or more CDK-4/6 inhibitors and/or one or more means to determine gene expression or mutation may be comprised in a kit. The kits will thus comprise, in suitable container means, one or more CDK-4/6 inhibitors and/or one or more means to determine gene expression and/or mutation.

The kits may comprise a suitably aliquoted one or more CDK-4/6 inhibitors and/or one or more means to determine gene expression or mutation of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the one or more CDK-4/6 inhibitors and/or one or more means to determine gene expression or mutation, and any other reagent containers in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

The kits of the present disclosure will also typically include a means for containing the vials in close confinement for commercial sale, such as, e.g., injection and/or blow-molded plastic containers into which the desired vials are retained.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### EXAMPLES

The following examples are included to demonstrate particular embodiments of the invention. It should be appreciated by those of skill in the art that the techniques disclosed in the examples which follow represent techniques discovered by the inventor to function well in the practice of the methods and compositions of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### NER and BER Downregulation Associates with Endocrine Treatment Resistance

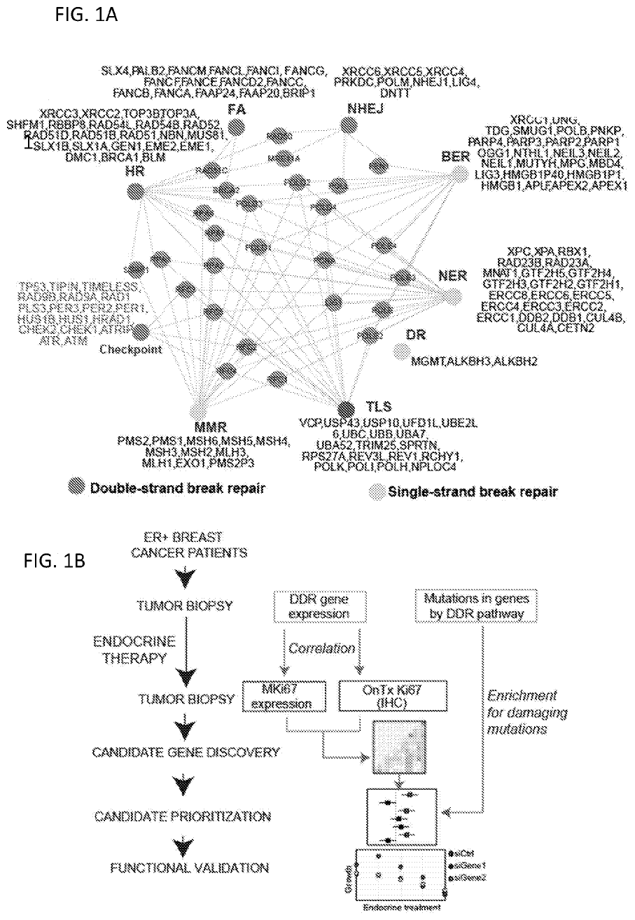

The status of 104 DDR genes belonging to the six major DDR pathways: NER, BER, MMR, NHEJ, FA and HR was assessed in primary ER+ breast tumors. Genes that were shared between multiple pathways were excluded from the analysis to facilitate better understanding of the discrete contributions of individual pathways to ET resistance (FIG. 1A).

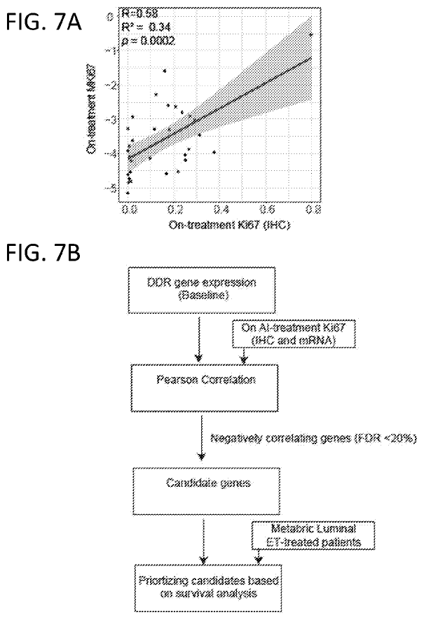

Additionally, direct repair (DR) and trans-lesion synthesis (TLS) were excluded because they fall outside canonical SSBR-DSBR categorization, and appeared to be rarely dysregulated in ER+ tumors in initial observations. The discovery set, referred to as NeoAI, was composed of data from two neoadjuvant aromatase inhibitor trials (Z1031(18, 19) and POL(20)). These clinical trials were designed to assess intrinsic ET response by accruing serial biopsies from patients at diagnosis (baseline, BL), after 2-4 weeks of endocrine treatment (on-treatment) and in the surgical specimen (FIG. 1B). These biopsies were annotated by whole genome/exome sequencing, RNA-seq and gene expression microarray, providing both mutational and transcriptomic information. The biopsies, both baseline and on-treatment were also evaluated for Ki67, a marker of proliferation, by both immunohistochemistry (IHC) and expression(21) (FIG. 7A). An on-treatment Ki67 value >10% is a clinically relevant marker of intrinsic ET resistance associated with elevated risk of relapse in the first 5 years of follow up (22). The discovery strategy for this study was therefore, to correlate DDR gene expression at baseline with on-treatment Ki67 levels (both by IHC and by expression), and combine results with that from a complementary enrichment analysis for deleterious mutations, to identify a set of DDR genes which when dysregulated may predict intrinsic ET resistance (FIG. 1B).

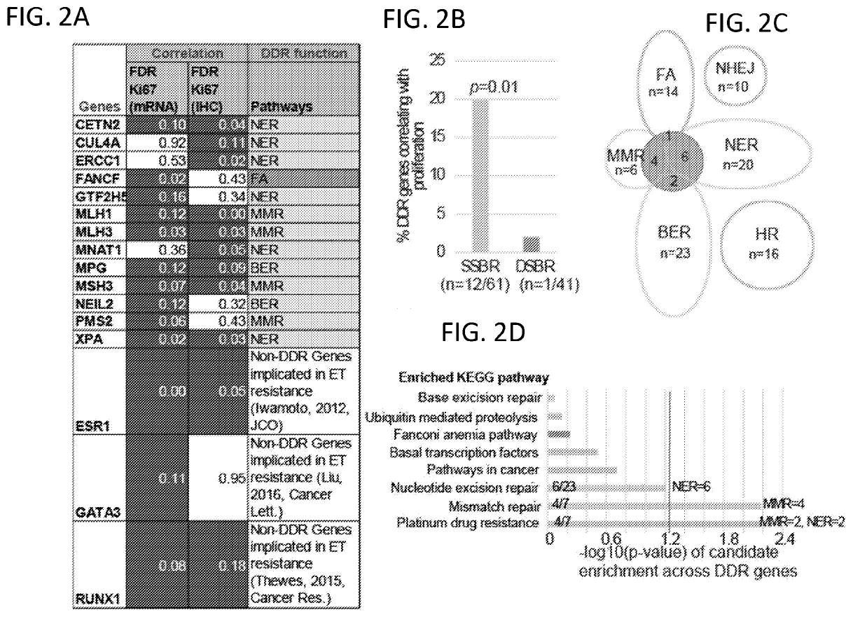

Transcriptomic analysis (schema outlined in FIG. 7B) identified 13 DDR genes whose reduced expression (at baseline) correlated with increased on-treatment Ki67 levels (FIG. 2A). Three genes (ESR1, GATA3 and RUNX1)(23-26) were used as positive controls because of their known associations with ET responsiveness (FIG. 2A). Twelve of the 13 DDR genes identified belonged to SSBR pathways, corresponding to ˜20% of all unique SSBR genes compared to only 2% of all unique DSBR genes (FIG. 2B) supporting a strong role for SSBR pathway downregulation in ET resistance. FA was the only one of three DSBR pathways examined to demonstrate significant association with ET resistance (FIG. 2C). Pathway enrichment analysis of the candidate gene list relative to all other DDR genes studied revealed significant over-representation of “Platinum drug resistance” (p=0.04, comprised of MMR and NER genes) and “mismatch repair” (p=0.04) terms (FIG. 2D). These results framed the proposition that underexpression of genes serving NER, and to a lesser extent, BER genes can reduce response to ET.

### Low-Expression of CETN2, ERCC1 and NEIL2 are Poor Prognostic Factors in ER+ Breast Cancer Patients Treated with Endocrine Therapy

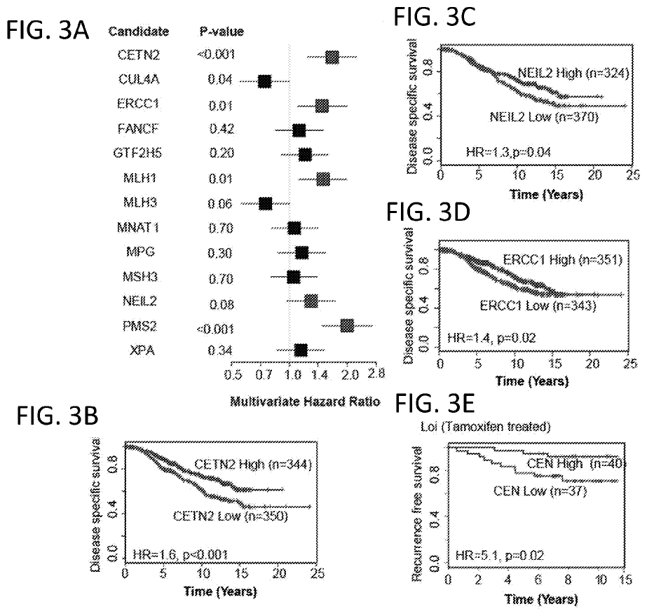

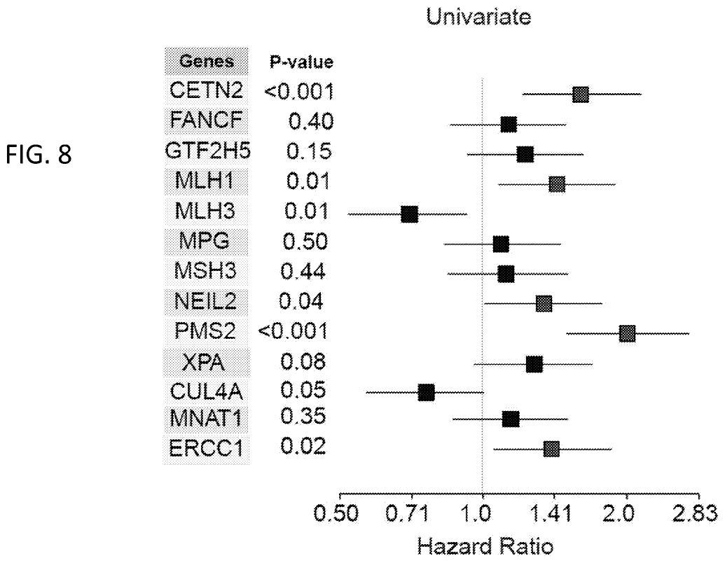

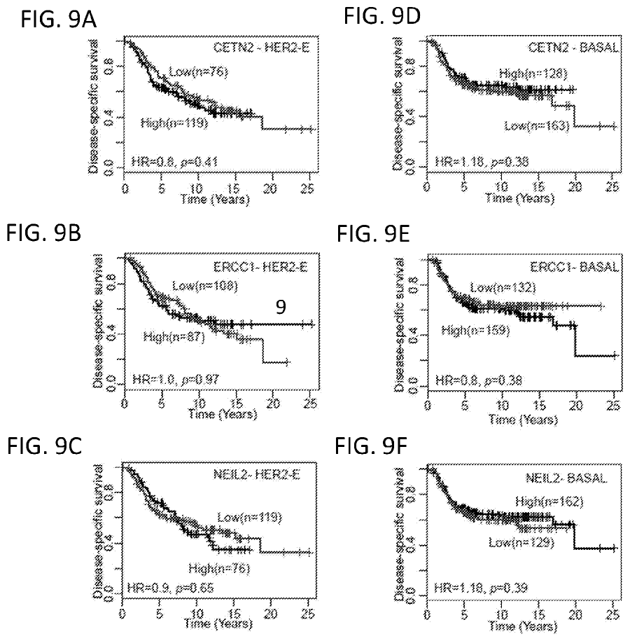

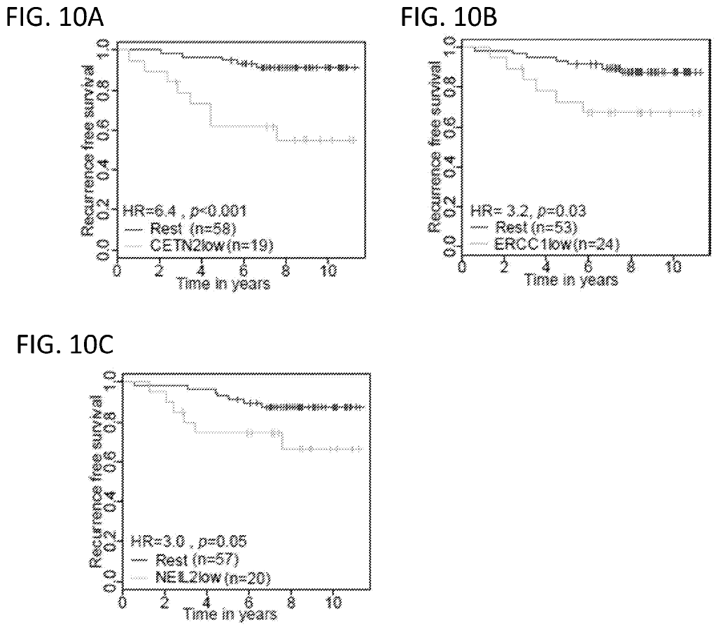

To understand the effect of downregulation of the 13 candidate genes on long-term patient outcomes, univariate associations between low-expression of these 13 genes and patient survival was tested in an independent data set (METABRIC). To increase the specificity of these correlations only the subset of patients with luminal tumors who were treated with ET was included in these analyses. Univariate Cox Proportional hazard analyses based on disease-specific survival identified five genes as significantly associating with poor survival: CETN2, ERCC1, MLH1, NEIL2 and PMS2 (FIG. 8). Subsequent multivariate analyses, including tumor size, grade and node status, supported an independent role for these five candidates in predicting disease-specific survival (FIG. 3A). An association between reduced expression of MutL genes, MLH1 and PMS2, and poor survival has already been described in this data set (12). Kaplan-Meier survival analysis demonstrated that low expression of CETN2, NEIL2, and ERCC1 individually also correlated with worse disease-specific survival compared to all other ER+ breast cancer patients (FIG. 3B-3D). However, no association between low RNA levels and poor survival for CETN2, NEIL2, and ERCC1 was observed in either HER2-enriched (FIG. 9A-9C) or basal-like (FIG. 9D-9F) breast cancer patients, suggesting that the association between defects in these genes and survival is subtype-specific. A correlation between low expression of CETN2, NEIL2 and ERCC1 and poor recurrence-free survival was also observed in the Loi data set (27), serving as independent validation (FIG. 10). Further, a composite signature based on low expression of any one of these genes (the CEN signature) associated with a significantly increased risk ratio of 5.1 in Loi (p=0.02, FIG. 3E).

### Damaging Mutations in Nucleotide Excision Repair, Base Excision Repair and Non-Homologous End Joining Genes are Enriched in ER+ Patient Tumors

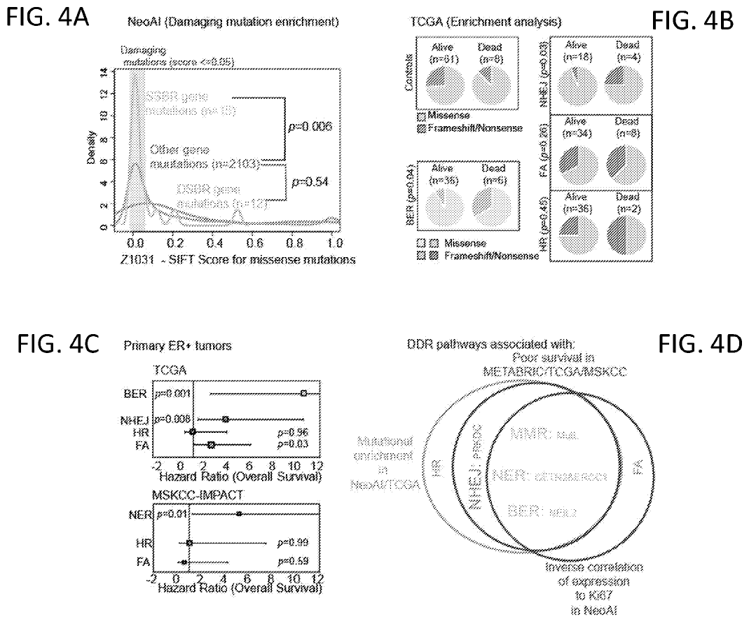

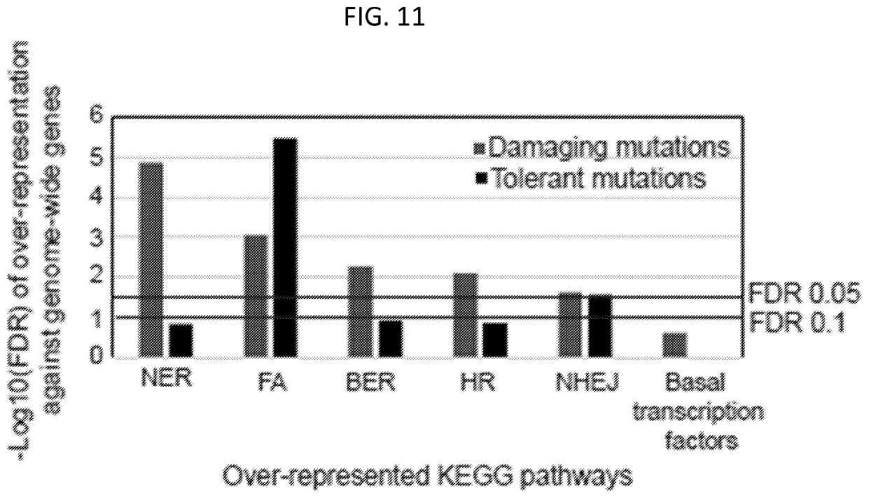

To further understand the involvement of DDR genes in ER+ breast cancer, incidence of damaging vs. non-damaging mis sense mutations (as predicted by SIFT (28, 29)) was analyzed in pre-treatment biopsies from NeoAI (Table 1). In this analysis, genes from SSBR pathways showed significant enrichment for damaging mutations when compared to all other genes in the genome whereas DSBR genes did not (FIG. 4A). Enrichment for damaging and tolerant mutations over genome-wide frequency was then assessed for each individual DDR pathway in NeoAI (FIG. 11).

TABLE 1
Involvement of DDR Genes in ER + Breast Cancer

| masked ID | gene | AAVar | pathway | Normal_VAF | Tumor_VAF | SIFT_ score |
|---|---|---|---|---|---|---|
| BRC10 | OGG1 | E103* | BER | 0 | 11.11 | NA |
| BRC10 | FANCE | R106W | FA | 0 | 13.68 | 0.25 |
| BRC14 | PARP4 | Q1059R | BER | 7.58 | 16.67 | 0.019 |
| BRC16 | CUL4A | E612K | NER | NA | NA | 0.044 |
| BRC32 | ERCC5 | R71S | NER | NA | NA | 0.01 |
| BRC37 | EME1 | E69D | HR | 8.4 | 48.31 | 1 |
| BRC39 | BRCA1 | S578F | HR | NA | NA | 0.008 |
| BRC47 | NEIL1 | A164S | BER | 0 | 23.08 | 0.061 |
| BRC50 | MLH3 | R1273T | MMR | NA | NA | 0.005 |
| BRC52 | PARP4 | Q1059R | BER | 8.51 | 18.03 | 0.019 |
| BRC7 | XRCC6 | M580I | NHEJ | NA | NA | 0.159 |
| CSB16 | NBN | K29R | HR | NA | NA | 0.048 |
| CSB22 | FANCM | K1215* | FA | NA | NA | NA |
| CSB22 | ERCC2 | G36fs | NER | NA | NA | NA |
| CSB26 | XPC | S255L | NER | NA | NA | 0.006 |
| CSB27 | LIG4 | E453Q | NHEJ | NA | NA | 0.005 |
| CSB3 | NHEJ1 | L115I | NHEJ | NA | NA | 0.125 |
| CSB6 | ERCC5 | M1041T | NER | NA | NA | 0.12 |
| CSB6 | ERCC2 | A3911 | NER | NA | NA | NA |
| BRCZ1 | PARP4 | I1039T | BER | 4.76 | 21.79 | 0.003 |
| BRCZ2 | DDB1 | E90K | NER | 0 | 10.14 | 0.209 |
| BRCZ3 | BLM | D44N | HR | 0 | 20.83 | 0.765 |
| BRCZ4 | SLX4 | H1049Y | FA | 0 | 40.38 | 0.411 |
| BRCZ5 | PARP2 | H5130 | BER | 0 | 14.29 | 0.526 |
| BRCZ6 | FANCG | A351E | FA | 0 | 13.04 | 0 |
| BRCZ7 | FANCD2 | S317N | FA | 0 | 18.75 | NA |
| BRCZ8 | NTHL1 | M102I | BER | 0 | 12.5 | 0.001 |
| BRCZ9 | SLX4 | E379K | FA | 0 | 10.29 | 0.168 |
| BRCZ10 | MLH3 | L1259P | MMR | 0 | 12.5 | 0.001 |
| BRCZ11 | FANCD2 | I1212L | FA | 0 | 11.76 | 0.74 |
| BRCZ12 | BLM | V1266A | HR | 0 | 23.19 | 0.005 |
| BRCZ13 | GTF2H4 | V56L | NER | 0 | 18.18 | 0.016 |
| BRCZ14 | PRKDC | L1850fs | NHEJ | 0 | 15.38 | NA |
| BRCZ15 | PARP4 | Q1059R | BER | 7.81 | 17.39 | 0.019 |

Damaging mutations were enriched in genes of NER, BER, NHEJ and HR pathways (FDR<0.05), but tolerant mutations were not, potentially indicating a selection for deleterious mutations in these pathways during tumor evolution. Damaging mutations were also enriched in genes of FA pathway but non-damaging mutations showed even higher enrichment, suggesting that any role for FA gene mutation in ER+ breast cancer is likely complex (FIG. 11). Enrichment for deleterious (frameshift/nonsense; FS/NS) over missense (MS) mutations in ER+ patient tumors was validated in TCGA for genes of BER and NHEJ, but not HR and FA, pathways, although due to limited follow-up time in this data set similar validation could not be obtained for NER (FIG. 4B). To facilitate rigorous statistical analysis, p-values were generated by comparing the proportion of somatic FS/NS:MS mutations in each DDR pathway in patients who were alive, to a control set of genes that have not been implicated as cancer drivers (SYNE1, MYH7, NEB). Together, these enrichment analyses promote the postulate that NER, BER and NHEJ gene mutations may be ER+ breast cancer drivers.

To address associations with clinical outcomes more directly, Cox Regression analysis was conducted for tumors with mutations in each DDR pathway in two data sets: TCGA and MSKCC-IMPACT (FIG. 4C). TCGA has whole exome sequence data from >800 ER+ breast tumors, while MSKCC-IMPACT has targeted sequencing of a selected panel of genes (including a subset of DDR genes) in >300 ER+ primary breast tumors. Amongst the SSBR pathways, mutations in NER (ERCC2-5) and BER genes each associated with significantly higher hazard ratio in MSKCC-IMPACT and TCGA databases, respectively (FIG. 4C), validating observations made in the gene expression (FIG. 2) and enrichment analyses described above (FIGS. 4A & 4B; FIG. 11). Association of NER gene mutations in TCGA and BER gene mutations in MSKCC-IMPACT could not be made because of median follow-up being <6 months in either case.

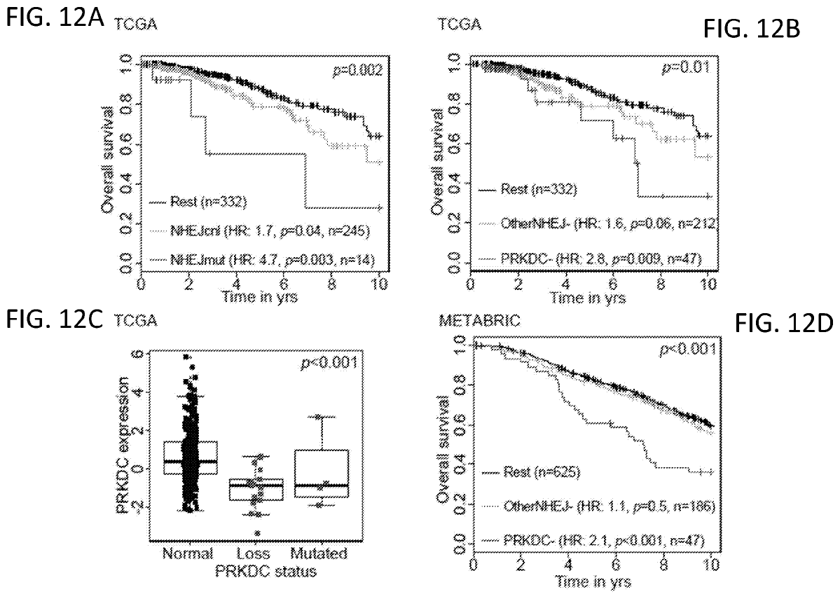

Amongst the DSBR pathways, tumors with mutations in NHEJ genes associated with a significantly higher hazard ratio when compared to wildtype tumors in TCGA (FIG. 4C). No NHEJ gene was included in the targeted panel sequenced in MSKCC-IMPACT precluding validation in this data set. To date, NHEJ has not been associated with ET resistance. Only five genes from this pathway had truncating mutations in either NeoAI or TCGA: PRKDC, XRCC5, DNTT, NHEJ1 and POLM. Patients whose tumors harbored either MS or FS/NS mutations in any of these genes associated with worse overall survival in TCGA, as did patients whose tumors had copy number loss of these loci (FIG. 12A). When individual associations with survival were analyzed, only mutation and/or copy number loss of PRKDC associated independently with poor survival (HR=2.8, p=0.009, FIG. 12B). Additionally, tumors with either PRKDC copy number loss or mutations also had significantly lower gene expression of PRKDC (p<0.001, FIG. 12C), suggesting that this is unlikely to be a chance association. Although mutation data on PRKDC was not available in METABRIC, the association of PRKDC copy number loss with poor survival was validated in METABRIC (FIG. 12D). Additionally, the association of PRKDC mutations with poor prognosis was recently observed in an independent set of ER+ patient tumors (30).

Amongst the other DSBR pathways, primary tumors with mutations in HR genes did not associate with higher hazard ratios than wildtype tumors in either TCGA or MSKCC-IMPACT where eight HR genes (RAD54L, RAD52, RAD51D, RAD51, NBN, BRCA1 and BLM) were included in the targeted panel (FIG. 4C). Mutations in FA genes had mixed associations, with patients whose tumors had mutations in FA genes associating with worse survival in TCGA but not in MSKCC-IMPACT (FANCA, FANCC, PALB2, BRIP1) (FIG. 4C).

Consideration of all three discovery parameters analyzed, i.e. gene expression downregulation, gene mutation, and association with patient outcomes (increased hazard ratios for overall survival in TCGA, METABRIC or MSKCC-IMPACT) provides strongest support for the involvement of SSBR pathway dysregulation in poor clinical outcomes of ER+ breast cancer patients (FIG. 4D). More specifically, all three discovery parameters support an understudied role for NER and BER dysregulation in ET resistance (FIG. 4D) that warrants functional investigation. Evidence for involvement of DSBR pathway dysregulation in ER+ breast cancer outcomes is less consistent across the three different screening parameters (FIG. 4D) and requires further investigation in larger patient cohorts, and in experimental model systems.

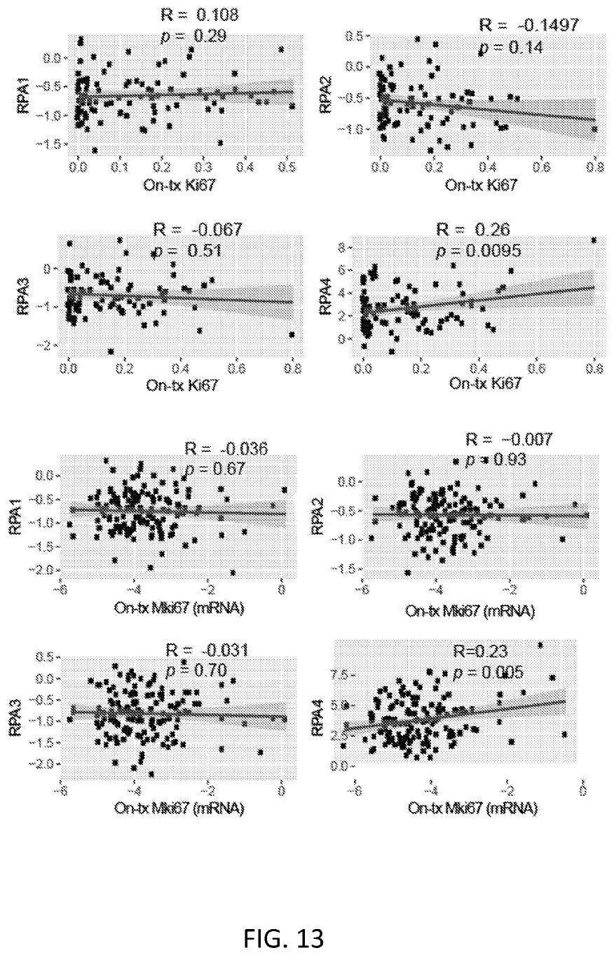

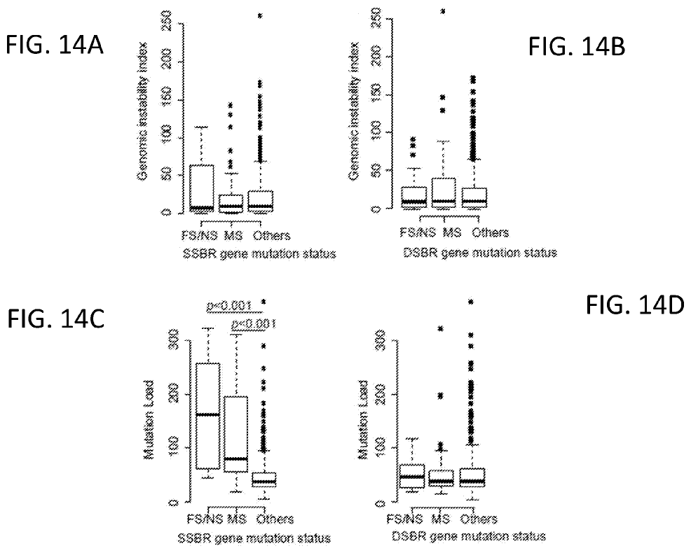

Two confounding factors affect interpretation of mutational analyses conducted here. Firstly, it is possible that dysregulation of replication factors commonly associated with DSBR disruption affects proliferative response to ET. However, no decisive association between replication gene expression (RPA1-4) and on-treatment proliferation marker Ki67 (IHC and mRNA, FIG. 13) was observed in NeoAI, suggesting that replicative disruption is unlikely to be a major confounding factor for this analysis. Secondly, previous reports have suggested an association between high mutation load or genome instability and poor patient outcomes in breast cancer (31), which may influence interpretation of associations between mutations in DDR genes and clinical outcome. No significant increase in genome instability or mutation load was observed in tumors with mutations in DSBR genes (FIG. 14B and FIG. 14D). However, as expected, a significant increase in mutation load, but not genome instability, was observed in SSBR mutated tumors (FIG. 14A and FIG. 14C). Therefore, it is not possible to rule out high mutation load as a potential confounding factor in the mutational analysis presented above, without functional validation of causality. Dysregulation of genes from the NER (CETN2 & ERCC1) and BER (NEIL2) pathways were, therefore, functionally investigated since candidates from these pathways were most consistently correlated with ET resistance and poor patient outcomes (FIG. 4D).

### Experimental Validation of CETN2, ERCC1 and NEIL2 as Endocrine Therapy Resistance Genes

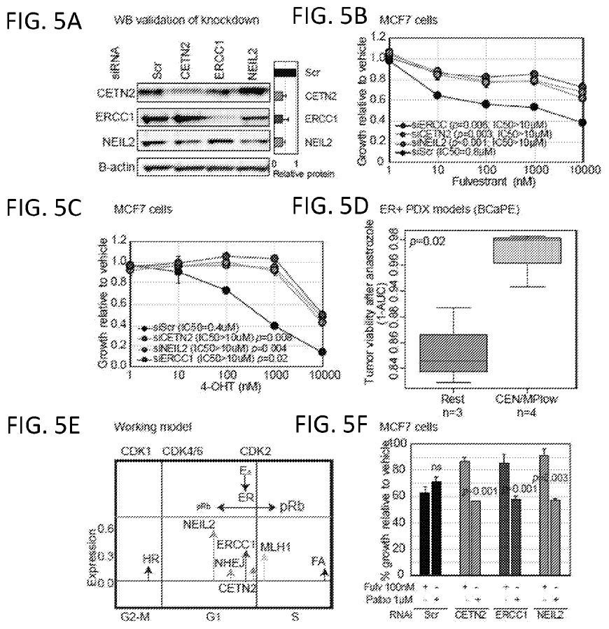

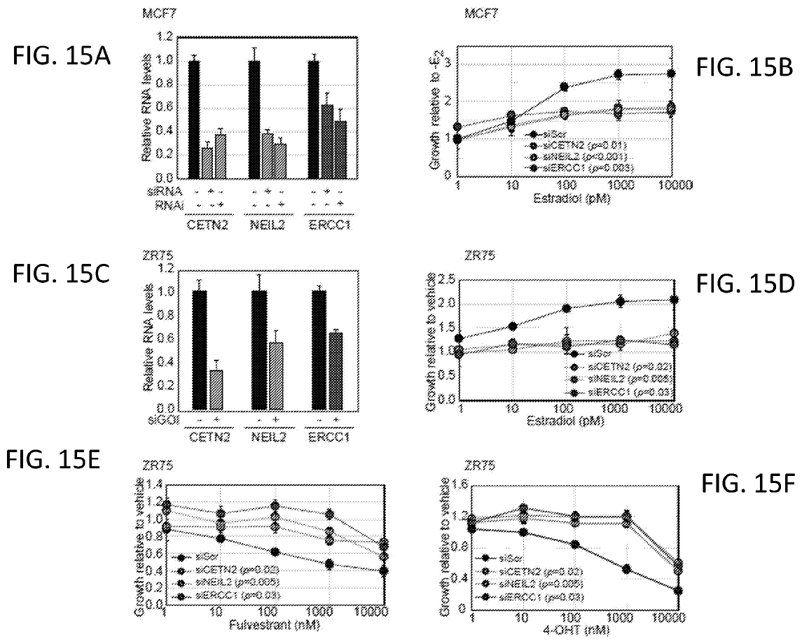

To test whether dysregulation of candidate genes from NER and BER pathways can directly cause ET resistance, pooled siRNA against each of the three candidate genes identified in the gene expression screen, i.e. CETN2, ERCC1 and NEIL2, as well as a scrambled control, was transiently transfected into two ET-sensitive, ER+ breast cancer cell lines, MCF7 (FIG. 5) and ZR-75 (FIG. 15). Knockdown of the genes was validated at RNA level in both cell lines (FIG. 15A & 15C), and downregulation of each protein was confirmed by Western blots of MCF7 cell lysates (FIG. 5A). Cells transfected with siRNA against scrambled control (siScr), CETN2, NEIL2 or ERCC1 were then exposed to all three classes of ET: estrogen deprivation in media containing charcoal stripped serum (to mimic AI), and tamoxifen or fulvestrant treatment in media containing full serum. Estrogen deprived siCETN2, siNEIL2, and siERCC1 MCF7 (FIG. 15B) and ZR-75 (FIG. 15D) cells showed attenulated growth response to estradiol stimulation when compared to siScr control cells, indicating decreased influence of estrogen signaling on proliferaton. Consistent with this notion, siCETN2, siNEIL2 and siERCC1 MCF7 (FIG. 5B & 5C) and ZR-75 (FIG. 15E & 15F) cells demonstrated a significant lack of growth inhibition in response to either fulvestrant or tamoxifen treatment.

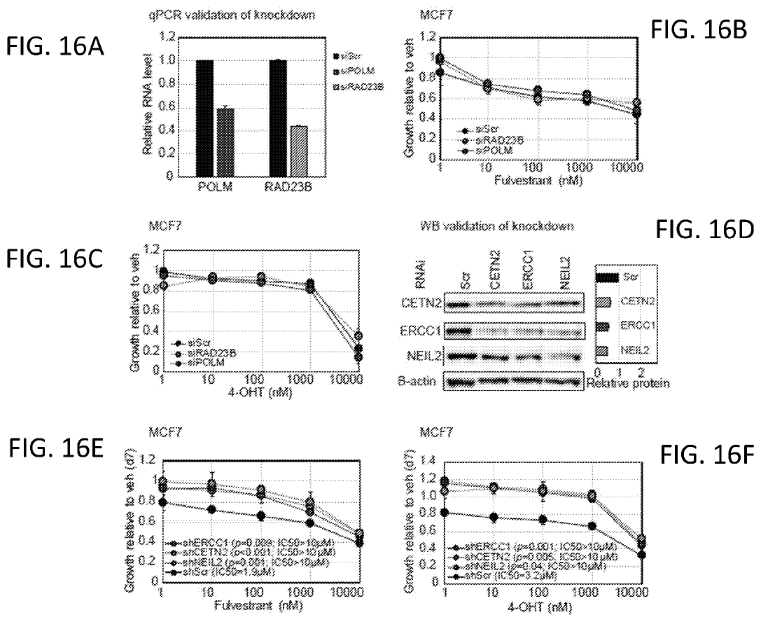

As negative controls, two DDR genes that did not correlate with ET resistance, RAD23B and POLM, were also knocked down using siRNA in MCF7 cells (FIG. 16A). Downregulation of these genes did not alter growth response to either fulvestrant (FIG. 16B) or tamoxifen (FIG. 16C). Additionally, independent lentiviral RNAi oligos against CETN2, NEIL2 and ERCC1 were used to stably select MCF7 cells with CETN2, ERCC1 and NEIL2 knockdown (FIG. 15A & 16D demonstrate knockdown at RNA and protein level respectively). Fulvestrant-independent (FIG. 10E) and tamoxifen-independent (FIG. 16F) growth phenotypes were faithfully replicated in these stable cells, providing orthogonal confirmation that growth effects caused by knockdown of the three candidate genes, CETN2, NEIL2 and ERCC1 are specific and causal.

Next, dysregulation of these candidates at either the mutational or RNA level was assessed across 7 ER+ PDXs (BCaPE(32)). One of the 7 lines had strong downregulation of NEIL2 expression, and two other lines exhibited downregulation of ERCC1. Additionally, one line had downregulation of PMS2 (MutL−). One of the lines with low ERCC1 RNA also harbored a missense mutation in MLH1 suggesting a compound phenotype. All four of these lines, (designated CENMP- because of a disruption of any CETN2, ERCC1, NEIL2 or MutL component) exhibited significantly higher tumor viability after treatment with the AI, anastrozole, when compared with the other three tumors designated CENMP+ in this PDX cohort (p=0.02, FIG. 5D).

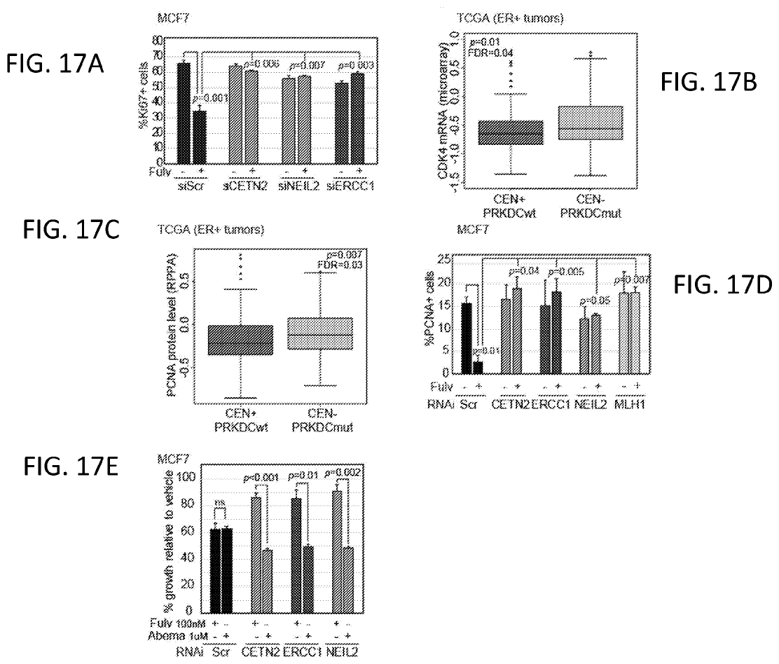

To test whether the loss of proliferative inhibition to ET observed in human tumors (FIG. 2) was reproducible in experimental systems, Ki67 levels were also assessed before and after fulvestrant treatment in siCETN2, siNEIL2 and siERCC1 MCF7 cells relative to siScr control. Inhibition of gene expression from any of these three candidates resulted in a profound lack of Ki67 inhibition in response to fulvestrant treatment, unlike control cells (FIG. 17A), reproducing observations in clinical trial samples. Earlier studies indicated that MutL-defective ER+ breast cancer cells exhibit altered proliferative response to ET due to dysregulation of G1/S cell cycle transition (12). To test whether the candidate DDR genes identified in this screen also participate in the regulation of G1/S transition, their gene expression pattern was analyzed across the cell cycle after a double thymidine block (www.dnarepairgenes.com (33)). All three candidates, as well as NHEJ genes, had maximal gene expression specifically in G1 or around the G1/S transition point, similarly to MLH1 (FIG. 5E). On the other hand, FA gene expression was maximal in late S phase and HR gene expression in late G2 (FIG. 5E). These observations are consistent with published data (34, 35) and indicate a common role for the candidate endocrine resistance DDR genes identified here in G1/S transition. As in the case of MutL-defective tumors, CEN/PRKDC− ER+ patient tumors from TCGA also had significantly increased RNA levels of CDK4, the principal G1 cyclin-dependent kinase (FIG. 17B) and protein levels of PCNA, a marker of successful S phase transition, relative to CEN/PRKDC+ tumors (FIG. 17C). Increased PCNA positivity was also confirmed in MCF7 cells with stable knockdown of CETN2, NEIL2, ERCC1 and MLH1 after fulvestrant treatment relative to control cells (FIG. 17D).

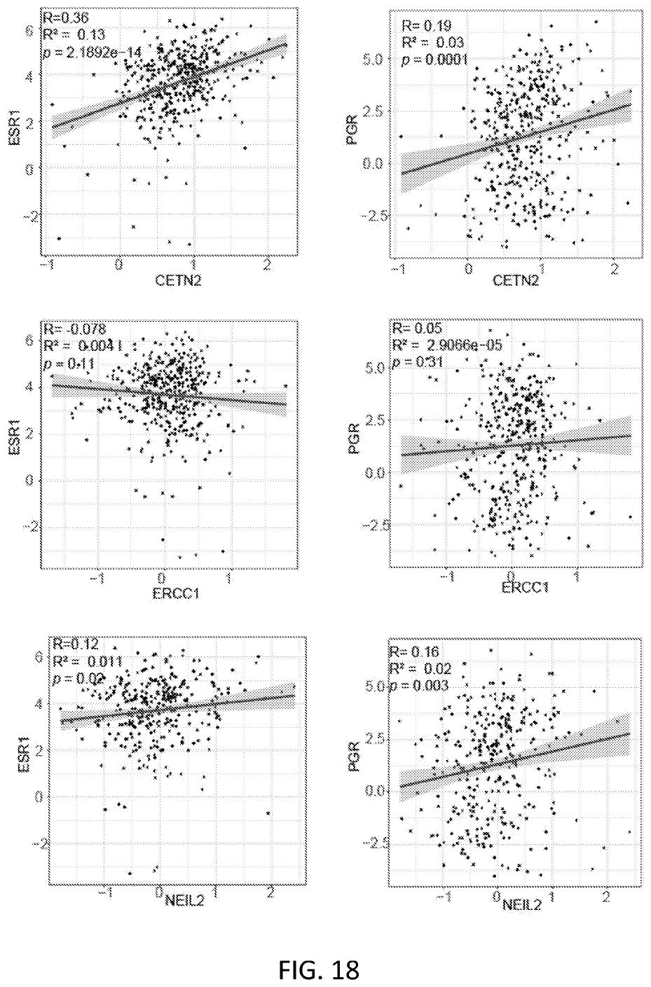

To test ER regulation as an alternative mechanism uniting these candidate genes in their ability to cause ET resistant growth, correlation of gene expression of each candidate gene was tested against ESR1/PGR expression in patient tumors from NeoAI (FIG. 18). Partial correlation was observed between ESR1/PGR levels and CETN2 (R=0.36/0.2), but not for other two genes. A ChIP-seq data set (36, 37) identified an ESR1 binding peak close to the CETN2 promoter but not for the other two candidates. Therefore, although ER-mediated regulation of some DDR genes cannot be ruled out as one mechanism underlying relationships with ET resistance, it is unlikely that it constitutes a common underlying mechanism for all the DDR candidate genes studies herein. Together, these data suggest that CEN− ER+ breast cancer cells, akin to MutL− cells, enable unchecked CDK4 activity, resulting in rapid G1/S transition even in the presence of ET.

To directly test whether inhibition of CDK4/6 can inhibit proliferation in CEN− ER+ breast cancer cells, MCF7 cells with stable knockdown of CETN2, NEIL2 or ERCC1 were exposed to the CDK4/6 inhibitors, palbociclib and abemaciclib. Control MCF7 cells demonstrated comparable sensitivity to both fulvestrant and CDK4/6 inhibitors, palbociclib (FIG. 5F) and abemaciclib (FIG. 17E), in keeping with published reports (12). However, downregulation of any one of the three candidate genes in MCF7 cells induced resistance to fulvestrant, but persistent sensitivity to both palbociclib (FIG. 5F) and abemaciclib (FIG. 17E). These data provide preliminary support for a role for DDR-dysregulation in predicting ET resistance and sensitivity to CDK4/6 inhibitors.

### Predictive Value of Candidate DNA Damage Repair Gene Dysregulation in ER+ Breast Cancer

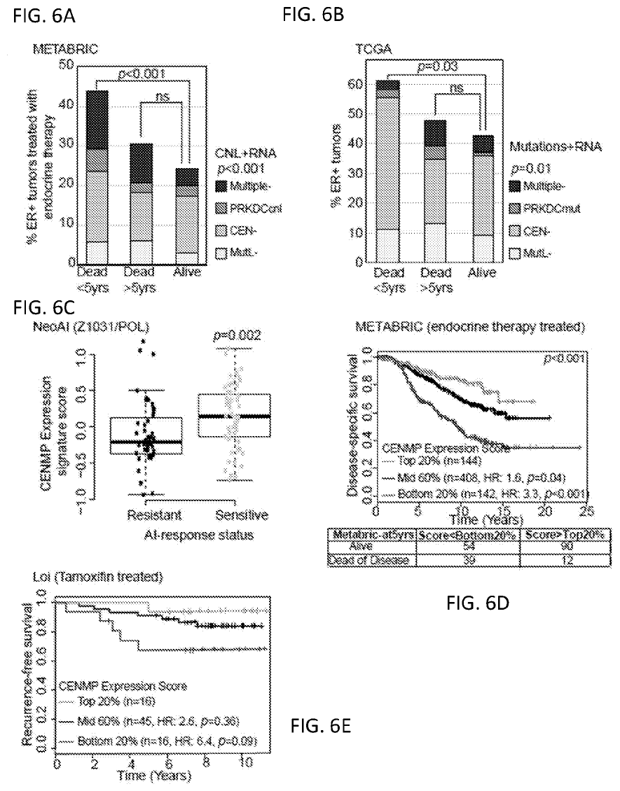

To estimate the impact of DDR dysregulation as a novel class of ET resistance driver and a predictive marker for ET failure, the cumulative frequency of dysregulation, i.e. multiple or co-occurring downregulation of 3 of the 4 novel candidate genes discovered in this analysis, CETN2, NEIL2, ERCC1, mutation or copy number loss of the fourth candidate gene, PRKDC, and downregulation of the 2 previously known candidate genes, MLHJ and PMS2 was assessed in METABRIC (FIG. 6A) and TCGA (FIG. 6B). In both data sets, downregulation of one or a combination of these genes occurred in 40-60% of tumors from ER+ breast cancer patients who died within 5 years of diagnosis. A less significant enrichment for dysregulation of these genes was observed in ER+ breast cancer patients who died more than 5 years after diagnosis, suggesting that downregulation of these genes predisposes ER+ breast cancer to early ET failure consistent with intrinsic resistance.

To identify a DDR-low signature in ER+ breast cancer patients, a gene expression score was defined using mean normalized expression of each gene. The score was significantly lower in resistant tumors from NeoAI when compared against sensitive counterparts (p=0.002, FIG. 6C). While this indicates that the score associates with ET resistance in patient tumors, the sensitivity of the score is ˜70% and the specificity ˜68%, indicating potential for further refinement of the signature by inclusion of other known factors, and mutational or copy number data.

Using this signature, the lowest and highest scoring quintiles of ER+ tumors were identified in METABRIC and Loi. The lowest scoring quintile associated with poor disease-specific and recurrence-free survival of patients with ER+ tumors in METABRIC (p<0.001, FIG. 6D) and Loi (p=0.09, FIG. 6E) indicating the feasibility of using this score to predict short-term outcomes in patient cohorts. Of note, this analysis also demonstrated better survival of patients in the upper quintile of the DDR signature score, suggesting dual validity of the score in predicting both worse and better response to ET.

### Significance of Certain Embodiments

This disclosure presents a comprehensive characterization of the molecular landscape of canonical DNA repair pathway defects in ER+ breast cancer as it relates to response to ET. A previous epidemiological study examining a selected subset of BER proteins using immunohistochemistry identified XRCC1, APE1, SMUG1, and FEN1 as associating with ER+ breast cancer specific survival (38).

The identification of DDR defects as regulators of response to ET also provide fundamental insights into into the etiology ER+breast cancer. Previous studies have identified lower incidence of structural rearrangements in ER+ breast tumors when compared to either ER− or HER2+ tumors(39). Simultaneously, whole exome sequencing identified a subset of ER+ tumors with high somatic mutation load as associating with poor survival, whereas high mutation load in ER− tumors trended towards an association with better patient survival(31). The ability of ER+ breast cancer cells to grow in the presence of SSBR defects may reflect the evolutionary context of normal ER+ mammary cells, which are primed for sudden and rapid bursts of proliferation, associated with downregulation of many SSBR pathways(14). In contrast, ER+ mammary cells may find it more difficult to tolerate large genomic rearrangements, commonly associated with DSBR defects, as this is not part of their etiology. Further analysis of the unique role of NHEJ loss in endocrine treatment response is warranted.

In terms of alternative therapeutic strategies for CENMP− ER+ breast cancer patients, this study provides some preliminary but potentially important associations that warrant deeper investigation. MutL-defective, ET resistant, ER+ breast cancer cells and tumors are sensitive to CDK4/6 inhibitors(12), currently in clinical use in advanced disease settings. Functional investigations presented herein extend these observations, indicating that a common mechanism underlying endocrine resistance caused by disruption of multiple DDR candidate genes from different pathways can generate a disconnect between ER and CDK4/6 that is targetable with CDK4/6 inhibition.

The CEN score, which takes into account MMR, BER and NER pathway genes is a new starting point for distinguishing patients into those who are not likely to respond to ET and will require alternative treatments potentially including CDK4/6 inhibition. However, sophisticated algorithms and inclusion of additional DDR genes and other known factors that regulate ET response may be utilized to improve the sensitivity and specificity of this signature, particularly for the prediction of ET response.

In summary, the results of this disclosure most clearly identify single-strand DNA damage repair defects as a novel class of ET resistance driver that in at least some embodiments contribute to perhaps half of ER+ breast cancer patient deaths within the first 5 years after diagnosis.

### Example 2

### Examples of Methods

### DDR Genes Set Compilation

Gene set for eight canonical DDR pathways comprising of Direct Repair (DR), Mismatch repair (MMR), Nucleotide excision repair (NER), Base excision repair (BER), homologous recombination (HR), non-homologous end joining (NHEJ) and Fanconi Anemia (FA) along with checkpoint genes was built as a union of MSigDB (40, 41) KEGG (c2:curated) pathway specific genes and updated table of DDR pathway genes listed at http://sciencepark.mdanderson.org/labs/wood/dna_repair_genes.html. Genes shared across different DDR pathways or checkpoints were not included in the analysis. Additionally, Translesion DNA synthesis (TLS) pathway genes were left out from current analysis, because of its ambiguous role in SSBR and DSBR mechanism. Cytoscape (42) was used to visualize the DDR pathway network.

### Patient Data

### Data Sets

Z1031/POL data set (referred to as NeoAI) was used with permission from the Alliance consortium. TCGA (downloaded: 06/17) and MSKCC-IMPACT (downloaded: 02/18) mutation data were obtained from cBio portal. TCGA (downloaded: 06/17) and METABRIC (downloaded: 06/17) copy number data were obtained from cBio portal. TCGA analyses were restricted to ER+ patient tumors (except for Figure S3 where basal-like and HER2-enriched tumors were analyzed based on published PAM50 categorization(43)). For MSKCC-IMPACT analyses, a list of ER− breast cancer sample IDs was derived from previously published literature (44) and subtracted from the list available on cBio Portal. While there is estimated to be some contamination from HER2+ or ER− breast cancer patients, this subtracted list contains a majority of ER+ breast tumors. TCGA and METABRIC gene expression data and associated survival outcomes were downloaded from Oncomine. Standard cut-offs of mean-1.5× standard deviation were used to identify “Low” subsets of each candidate gene in each data set when multiple candidate genes were combinatorially analyzed. For individual analyses in METABRIC, “Low” subsets were identified using median cut-offs, and in Loi, using mean-1.5× standard deviation.

### Enrichment Analysis

Mutations from NeoAI (18, 20) and were used as the discovery set along with DNA-sequenced ER+ tumors from TCGA. For NeoAI data set, mutations with Normal VAF<10/NA and Tumors VAF>10/NA were classified as somatics. Mutations were scored by SIFT (28, 29) to assess the effect on the protein structure and function. In accordance with SIFT standards, missense mutations with scores <0.05 were considered to be damaging. For the enrichment analysis in TCGA, mutations were categorized into three variant types—Missense, frameshift and nonsense. Frameshift and nonsense mutations were cumulatively referred to as FS/NS mutations. Enrichment analysis in TCGA used the Z-score test of two population proportions to compare the proportion of missense to frameshift/nonsense mutations in each DDR pathway to the proportion of mis sense to frameshift/nonsense mutations in a control set of unrelated genes (MYH7, SYNE1, NEB) in tumors from patients who remained alive. This approach was used to ensure the presence of appropriate sample size for the analysis employed and to control for genome-wide levels of mutations in each tumor.

### Survival Analysis

For the univariate and multivariate analysis, we analyzed 887 tumors from Luminal (A/B) patients who received endocrine treatment. mRNA (microarray) expression and survival information along with other clinical metadata were extracted from Oncomine (45-47). Only samples with survival metadata were included in the analysis. For this cohort, tumors with expression level of candidate genes lower than median values were labeled as “low” while rest were labelled as “high”. All survival data was analyzed using Kaplan-Meier curves and log-rank tests. Proportional hazards were determined using Cox regression.

### Statistical Analysis

Missing data were imputed with “NA” from mutation, expression and survival data analysis. Samples classifying for more than one category were either removed (e.g. samples with mutations in both SSBR and DSBR genes) or treated as separate set (e.g. cumulative analysis of tumors with dysregulation of new versus published candidates) for statistical comparisons. Pearson's correlation was performed on the log transformed normalized data for every DDR gene using automated script in R. To control errors in multiple test corrections, false discovery rates were calculated using the Benjamini-Hochberg (48) method in R. Two-tailed Wilcoxon rank sum tests were used for two-sample tests of association between classes. Pathway over-representation analysis was performed using thirteen candidate genes as the input genes against all DDR genes (n=104) as the background in WebGestalt (49) using KEGG pathway database.

An expression signature score known as CENMP (CETN2,ERCC1, NEIL2, MLH1, PMS2) score was devised using mean of standardized expression for the mentioned fives genes. The CENMP score was calculated for the three independent data set (NeoAI, Metabric and Loi) using the microarray expression levels. Survival of patients with highest 20% quantile of CENMP score was compared against that of patients with lowest 20% quantile of the same.

### Cell Lines, siRNA Transfection and Growth Assays

ZR75.1 and MCF7 parental cells were from ATCC, 2015 and 2017 respectively, and tested for mycoplasma contamination upon arrival using the Lonza Mycoalert Plus Kit (CAT# LT07-710) as per the manufacturer's instructions, and annually since. Both lines were maintained in RPMI 1640 1X w/ L-glutamine and 1% Penicillin/Streptomycin (Sigma-Aldrich, CAT#P4333—100 mL). Cell lines used for experiments were <20 passages. Transient transfections with esiRNA (Sigma-Aldrich) towards human NEIL2 (CAT#EHU158461), CETN2 (CAT#EHU137031), ERCC1 (CAT#EHU156971), RAD23B (CAT#EHU145881) and POLM (CAT#SASI_Hs02_003344553), or scrambled control used at 50 nmoles/L each were delivered using Polypluys jetPRIME® transfection reagent (CAT#114-07) as per the manufacturer's instructions. Cells were plated for experiments 48 hrs after transfection. Stable selection with puromycin after infection with lentivirus harboring RNAi oligos (ABM) against human NEIL2 (CAT#i014980a), CETN2 (CAT#i004479a), and ERCC1 (CAT#i007023a) or scrambled control (CAT#i000238c) was conducted using manufacturer's protocol. Growth assays were repeated independently in triplicate as previously reported (50) using Alamar blue to detect cell viability. Final readings were between 4-6 days after initial drug treatment and fold change plotted for analysis.

### Drug-Treatment

Fulvestrant(Fisher Scientific CAT#506242), 4-OHT(Sigma-Aldrich CAT#H7904), Palbociclib (ThermoFisher CAT#508548) and Abemaciclib (ThermoFisher CAT#NC0577560), dissolved in DMSO, and β-estradiol (Sigma-Aldrich CAT#E2758-1G), dissolved in water, at 10 mmoles/L were stored long term at −80 C with working stocks at −20 C. Cells were treated 24 hours after plating with fresh drug added every 48 hrs until the end of the experiment. Estradiol addition experiments were conducted in phenol red-free DMEM with 10% charcoal stripped serum.

### qPCRs, Western Blot, FACS and IFs

RNA from cell lines was extracted using the Qiagen RNeasy Mini Kit (CAT#74106) and converted to cDNA using Bio-Rad Reverse Transcriptase iScript (CAT#TX1708891BAY), both following manufacturers' instructions. cDNA was quantified by qPCR using Bio-Rad SsoAdvanced Universal SYBR Green supermix (CAT#17525272) at the manufacturers specifications. For Immunoflourescence, 48 hrs after esiRNA transfection cells were plated on Poly-D-Lysine coated coverslips (CAT#NC0746078), treated for 24 hrs, then harvested. Cells on coverslips were then washed with 1× PBS, fixed in 4% PFA, and then co-stained with Ki-67/MKI67 Antibody (Novus-Biologicals CAT#NB110-89717SS). Western blotting was conducted as described previously (50) using antibodies against CETN2 (ABclonal CAT#A5397), NEIL2 (Abcam CAT#ab221556), ERCC1 (Abcam CAT#ab129267), and β-Actin (Sigma-Aldrich CAT#A5316—100 ul). FACS analysis for PCNA (Abcam CAT#ab29) was conducted after treating MCF7 siScr or siCEN cells with 100 nM Fulvestrant for 72 hours following a 20-hour treatment with 250 nM nocodazole (Sigma-Aldrich CAT#M1404). Cells were then probed for PCNA and run on a BD Accuri C6 flow cytometer using a standard protocol.

### Example 3

### Studies in ER+ Bladder and Colorectal Cancer

Significance: Mutational inactivation and/or low RNA levels of the MutL (MLH1, PMS1, PMS2) complex of mismatch repair (MMR) is a contributor to endocrine therapy resistance in ˜15% of estrogen receptor positive (ER+) breast cancer patients(1). The work identified a sharp dichotomy within the MMR pathway in terms of breast cancer prognosis, with MutS complex (MSH2-6) associating with better survival. Several other solid cancers also have high frequency of MMR loss, including bladder (22%) and colorectal cancer (CRC) (27%)(2-4). Individual roles of MutL vs MutS complexes in these cancer types are understudied, but MMR dysregulation as a whole is considered a good prognostic factor(5). This observation may be driven by MutS associations. Indeed, analysis of TCGA datasets indicates that MutL loss in both bladder and CRC associates with significantly worse patient survival, although MutS loss does not. This finding challenges the existing paradigm that MMR loss is a good prognostic factor, and suggests an alternate hypothesis that MutL and MutS loss must be considered individually as prognostic factors in cancer.

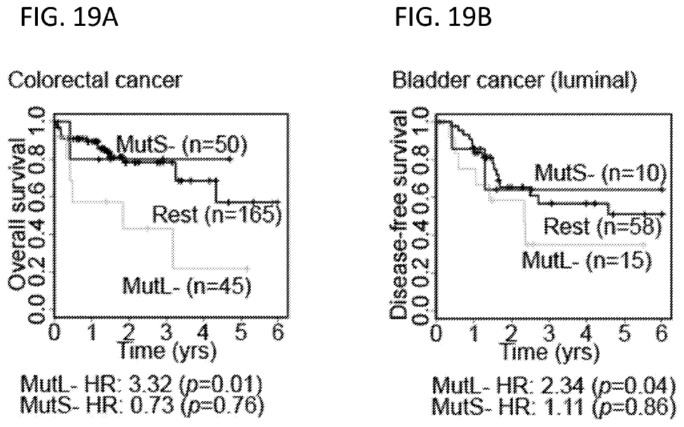

MMR is the primary replication error repairing pathway. In absence of MMR, point mutations and indels increase with every replicative cycle(6,7), eventually resulting in catastrophic cell cycle arrest and death(8). Somatic MutL mutations occur in at least 5% of stomach, lung, endometrial, bladder and CRC. This is in keeping with previously published results as many of these cancer types are part of the Lynch syndrome spectrum, characterized by germline mutations in both MutL and MutS genes(9). Increased somatic mutational frequency of MutL over MutS genes is common to only three cancer types: ER⁺ breast, bladder and pancreas, while archetypal Lynch syndrome cancers e.g. CRC, endometrial, have similar incidence of both MutL and MutS somatic gene mutations. Both bladder and CRC are difficult to treat and are associated with high mortality rates(10-13). By and large, MMR dysregulation in these cancer types has been considered as a whole, without comprehensive dichotomization into MutL vs MutS dysregulated tumors(14-17). In this context, MMR-loss in both bladder and CRC has been so far associated with relatively good prognosis. Analysis of TCGA data indicates that MutL, but not MutS, loss, by mutation or low RNA levels, associates with significantly worse survival in both bladder and CRC (FIGS. 19A and 19B). Of note, the association with worse survival in bladder cancer is restricted to the luminal subset, a molecular subtype similar to ER+ breast cancer(18,19). Luminal bladder cancer is enriched for muscle-invasive pathology, and associated with aggressive presentation & poor clinical outcomes(20,21).

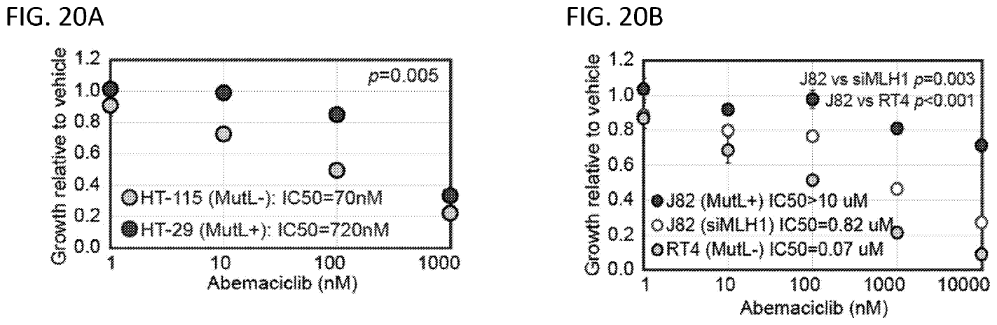

Recently, there has been increased interest in treating ER⁺ breast, colorectal and bladder cancers with CDK4/6 inhibitors. However, additional predictive markers are required for successful administration of these inhibitors, as they only work in as yet incompletely defined subsets of tumors(22)(23). Impact of MutL defects on CDK4/6 addiction has not been reported, but a role for MutL-loss in predicting response to CDK4/6 inhibitors is plausible based on the finding that MutL-loss is a predictor of response to CDK4/6 inhibitors in ER⁺ breast cancer in vitro, in vivo and in patient tumors(1). Studies indicate that intrinsically MutL⁻ CRC line, HT-115 and bladder cancer cell line, RT4, are more susceptible to CDK4/6 inhibitor, abemaciclib, than MutL⁺ cells (FIG. 20). Moreover, knocking down MLH1 with pooled siRNA in MutL⁺ bladder cancer cell line, J-82 induces sensitivity to abemaciclib (FIG. 20B). TCGA RPPA data confirms significant upregulation of CCND1/D3, CDK4 partners, in ER⁺ breast, bladder and colorectal MutL⁻ vs MutL⁺ tumors (p<0.001; FDR<0.01), indicating relevance to human tumors.

These data indicate a role for the four principal MutL genes: MLH1, MLH3, PMS1 and PMS2, in predicting resistance to standard-of-care, and most importantly, sensitivity to CDK4/6 inhibitors. Diagnostic tests for this combination of genes can be used to identify patients most likely to be resistant to standard-of-care and likely to respond well to CDK4/6 inhibitors.

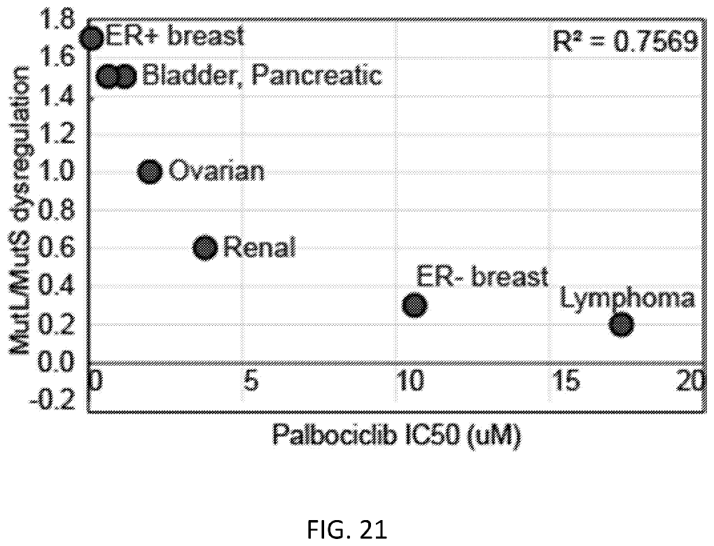

FIG. 21 illustrates an inverse correlation between frequency of MutL dysregulation and response to CDK-4/6 inhibitor, Palbociclib. ER+ breast, bladder, pancreatic, and ovarian cancer types that have increased frequency of MutL over MutS dysregulation are more like to be sensitive to Palbociclib (as an example of a CDK-4/6 inhibitor) than cancer types that have predominant MutS dysregulation, i.e. ER- breast and lymphoma.

### References for Example 3

1. Haricharan S, Punturi N, Singh P, Holloway K R, Anurag M, Schmelz J, et al. Loss of MutL Disrupts Chk2-dependent Cell Cycle Control Through CDK4/6 to Promote Intrinsic Endocrine Therapy Resistance in Primary Breast Cancer. Cancer Discov. 2017;

2. Rubio J, Blanes A, Sanchez-Carrillo J J, Diaz-Cano S J. Microsatellite abnormalities and somatic down-regulation of mismatch repair characterize nodular-trabecular muscle-invasive urothelial carcinoma of the bladder. Histopathology. 2007;51:458-67.

3. Donehower L A, Creighton C J, Schultz N, Shinbrot E, Chang K, Gunaratne P H, et al. MLH1-silenced and non-silenced subgroups of hypermutated colorectal carcinomas have distinct mutational landscapes. J Pathol. 2013;229:99-110.

4. Hewish M, Lord C J, Martin S A, Cunningham D, Ashworth A. Mismatch repair deficient colorectal cancer in the era of personalized treatment. Nature Reviews Clinical Oncology. 2010;7:197-208.

5. Alex A K, Siqueira S, Coudry R, Santos J, Alves M, Hoff P M, et al. Response to Chemotherapy and Prognosis in Metastatic Colorectal Cancer With DNA Deficient Mismatch Repair. Clinical Colorectal Cancer [Internet]. 2016 [cited 2017 Mar. 23];0. Available from: http://www.clinical-colorectal-cancer.com/article/S1533-0028(16)30255-9/abstract

6. Baross-Francis A, Andrew S E, Penney J E, Jirik F R. Tumors of DNA mismatch repair-deficient hosts exhibit dramatic increases in genomic instability. Proc Natl Acad Sci USA. 1998;95:8739-43.

7. Narayanan L, Fritzell J A, Baker S M, Liskay R M, Glazer P M. Elevated levels of mutation in multiple tissues of mice deficient in the DNA mismatch repair gene Pms2. Proc Natl Acad Sci USA. 1997;94:3122-7.

8. Liu A, Yoshioka K-I, Salerno V, Hsieh P. The mismatch repair-mediated cell cycle checkpoint response to fluorodeoxyuridine. J Cell Biochem. 2008;105:245-54.

9. Win A K, Lindor N M, Winship I, Tucker K M, Buchanan D D, Young J P, et al. Risks of colorectal and other cancers after endometrial cancer for women with Lynch syndrome. J Natl Cancer Inst. 2013;105:274-9.

10. El Bairi K, Kandhro A H, Gouri A, Mahfoud W, Louanjli N, Saadani B, et al. Emerging diagnostic, prognostic and therapeutic biomarkers for ovarian cancer. Cell Oncol (Dordr). 2017;40:105-18.

11. Pishvaian M J, Brody J R. Therapeutic Implications of Molecular Subtyping for Pancreatic Cancer. Oncology (Williston Park, N.Y.). 2017;31.

12. Sharma P, Zargar-Shoshtari K, Pow-Sang J M. Biomarkers for prostate cancer: present challenges and future opportunities. Future Sci OA. 2016;2:FSO72.

13. Mármol I, Sánchez-de-Diego C, Pradilla Dieste A, Cerrada E, Rodriguez Yoldi M J. Colorectal Carcinoma: A General Overview and Future Perspectives in Colorectal Cancer. Int J Mol Sci. 2017;18.

14. Catto J W F, Xinarianos G, Burton J L, Meuth M, Hamdy F C. Differential expression of hMLH1 and hMSH2 is related to bladder cancer grade, stage and prognosis but not microsatellite instability. Int J Cancer. 2003;105:484-90.

15. Mylona E, Zarogiannos A, Nomikos A, Giannopoulou I, Nikolaou I, Zervas A, et al. Prognostic value of microsatellite instability determined by immunohistochemical staining of hMSH2 and hMSH6 in urothelial carcinoma of the bladder. APMIS. 2008;116:59-65.

16. Clark A J, Barnetson R, Farrington S M, Dunlop MG. Prognosis in DNA mismatch repair deficient colorectal cancer: are all MSI tumours equivalent? Fam Cancer. 2004;3:85-91.

17. Ghanipour L, Jirström K, Sundstrom M, Glimelius B, Birgisson H. Associations of defect mismatch repair genes with prognosis and heredity in sporadic colorectal cancer. Eur J Surg Oncol. 2017;43:311-21.

18. Mitra A P. Molecular sub stratification of bladder cancer: moving towards individualized patient management. Ther Adv Urol. 2016;8:215-33.

19. Kim J, Akbani R, Creighton C J, Lerner S P, Weinstein J N, Getz G, et al. Invasive Bladder Cancer: Genomic Insights and Therapeutic Promise. Clin Cancer Res. 2015;21:4514-24.

20. Warrick JI, Walter V, Yamashita H, Chung E, Shuman L, Amponsa V O, et al. FOXA1, GATA3 and PPAR [symbol] Cooperate to Drive Luminal Subtype in Bladder Cancer: A Molecular Analysis of Established Human Cell Lines. Sci Rep [Internet]. 2016;6. Available from: http://www.ncbi.nlm.nih.gov/pmc/articles/PMC5141480/

21. Blanes A, Rubio J, Sanchez-Carrillo J J, Diaz-Cano S J. Coexistent intraurothelial carcinoma and muscle-invasive urothelial carcinoma of the bladder: clonality and somatic down-regulation of DNA mismatch repair. Hum Pathol. 2009;40:988-97.

22. Lee M S, Helms T L, Feng N, Gay J, Chang Q E, Tian F, et al. Efficacy of the combination of MEK and CDK4/6 inhibitors in vitro and in vivo in KRAS mutant colorectal cancer models. Oncotarget. 2016;7:39595-608.

23. Sathe A, Koshy N, Schmid S C, Thalgott M, Schwarzenböck S M, Krause B J, et al. CDK4/6 Inhibition Controls Proliferation of Bladder Cancer and Transcription of RB1. J Urol. 2016;195:771-9.

### REFERENCES

The entire contents of all patents, published patent applications, and other references cited herein are hereby expressly incorporated herein in their entireties by reference.

1. Davies C, et al. (2011) Relevance of breast cancer hormone receptors and other factors to the efficacy of adjuvant tamoxifen: patient-level meta-analysis of randomised trials. Lancet (London, England) 378(9793):771-784.

2. Ma C X, Reinert T, Chmielewska I, & Ellis M J (2015) Mechanisms of aromatase inhibitor resistance. Nature reviews. Cancer 15(5):261-275.

3. Bose R, et al. (2013) Activating HER2 mutations in HER2 gene amplification negative breast cancer. Cancer discovery 3(2):224-237.

4. Slamon D J, et al. (1987) Human breast cancer: correlation of relapse and survival with amplification of the HER-2/neu oncogene. Science (New York, N.Y.) 235(4785):177-182.

5. Yersal O & Barutca S (2014) Biological subtypes of breast cancer: Prognostic and therapeutic implications. World Journal of Clinical Oncology 5(3):412-424.

6. Goncalves R, Ma C, Luo J, Suman V, & Ellis M J (2012) Use of neoadjuvant data to design adjuvant endocrine therapy trials for breast cancer. Nature reviews. Clinical oncology 9(4):223-229.

7. Cheang M C U, et al. (2009) Ki67 Index, HER2 Status, and Prognosis of Patients With Luminal B Breast Cancer. JNCI Journal of the National Cancer Institute 101(10):736-750.

8. Barone I, et al. (2009) Expression of the K303R estrogen receptor-alpha breast cancer mutation induces resistance to an aromatase inhibitor via addiction to the PI3K/Akt kinase pathway. Cancer research 69(11):4724-4732.

9. Osborne C K & Schiff R (2011) Mechanisms of endocrine resistance in breast cancer. Annual review of medicine 62:233-247.

10. de Groot A F, Kuijpers C J, & Kroep J R (2017) CDK4/6 inhibition in early and metastatic breast cancer: A review. Cancer Treat Rev 60:130-138.

11. Kwapisz D (2017) Cyclin-dependent kinase 4/6 inhibitors in breast cancer: palbociclib, ribociclib, and abemaciclib. Breast cancer research and treatment 166(1):41-54.

12. Haricharan S, et al. (2017) Loss of MutL Disrupts CHK2-Dependent Cell-Cycle Control through CDK4/6 to Promote Intrinsic Endocrine Therapy Resistance in Primary Breast Cancer. Cancer discovery 7(10):1168-1183.

13. Broustas C G & Lieberman H B (2014) DNA Damage Response Genes and the Development of Cancer Metastasis. Radiation research 181(2):111-130.

14. Caldon C E (2014) Estrogen signaling and the DNA damage response in hormone dependent breast cancers. Frontiers in oncology 4:106.

15. Nik-Zainal S, et al. (2012) Mutational Processes Molding the Genomes of 21 Breast Cancers. Cell 149(5-10):979-993.

16. Dietlein F, Thelen L, & Reinhardt H C (2014) Cancer-specific defects in DNA repair pathways as targets for personalized therapeutic approaches. Trends in genetics: TIG 30(8):326-339.

17. Nickoloff J A, Jones D, Lee S H, Williamson E A, & Hromas R (2017) Drugging the Cancers Addicted to DNA Repair. Journal of the National Cancer Institute 109(11).

18. Ellis M J, et al. (2011) Randomized phase II neoadjuvant comparison between letrozole, anastrozole, and exemestane for postmenopausal women with estrogen receptor-rich stage 2 to 3 breast cancer: clinical and biomarker outcomes and predictive value of the baseline PAM50-based intrinsic subtype--ACOSOG Z1031. Journal of clinical oncology: official journal of the American Society of Clinical Oncology 29(17):2342-2349.

19. Ellis M J, et al. (2012) Whole-genome analysis informs breast cancer response to aromatase inhibition. Nature 486(7403):353-360.

20. Olson J A, Jr., et al. (2009) Improved surgical outcomes for breast cancer patients receiving neoadjuvant aromatase inhibitor therapy: results from a multicenter phase II trial. Journal of the American College of Surgeons 208(5):906-914; discussion 915-906.

21. Ellis M J, et al. (2017) Ki67 Proliferation Index as a Tool for Chemotherapy Decisions During and After Neoadjuvant Aromatase Inhibitor Treatment of Breast Cancer: Results From the American College of Surgeons Oncology Group Z1031 Trial (Alliance). Journal of clinical oncology: official journal of the American Society of Clinical Oncology 35(10):1061-1069.

22. Urruticoechea A, Smith I E, & Dowsett M (2005) Proliferation marker Ki-67 in early breast cancer. Journal of clinical oncology: official journal of the American Society of Clinical Oncology 23(28):7212-7220.

23. Liu J, et al. (2016) GATA3 mRNA expression, but not mutation, associates with longer progression-free survival in ER-positive breast cancer patients treated with first-line tamoxifen for recurrent disease. Cancer Lett 376(1):104-109.

24. Chimge N O, et al. (2016) RUNX1 prevents oestrogen-mediated AXIN1 suppression and beta-catenin activation in ER-positive breast cancer. Nat Commun 7:10751.

25. Thewes V, et al. (2015) Reprogramming of the ERRalpha and ERalpha target gene landscape triggers tamoxifen resistance in breast cancer. Cancer research 75(4):720-731.

26. Iwamoto T, et al. (2012) Estrogen receptor (ER) mRNA and ER-related gene expression in breast cancers that are 1% to 10% ER-positive by immunohistochemistry. Journal of clinical oncology: official journal of the American Society of Clinical Oncology 30(7):729-734.

27. Loi S, et al. (2008) Predicting prognosis using molecular profiling in estrogen receptor-positive breast cancer treated with tamoxifen. BMC genomics 9:239.

28. Ng P C & Henikoff S (2003) SIFT: Predicting amino acid changes that affect protein function. Nucleic acids research 31(13):3812-3814.

29. Liu X, Jian X, & Boerwinkle E (2011) dbNSFP: a lightweight database of human nonsynonymous SNPs and their functional predictions. Hum Mutat 32(8):894-899.

30. Griffith O L, et al. (2017) The prognostic effects of somatic mutations in ER-positive breast cancer. bioRxiv.

31. Haricharan S, Bainbridge M N, Scheet P, & Brown P H (2014) Somatic mutation load of estrogen receptor-positive breast tumors predicts overall survival: an analysis of genome sequence data. Breast cancer research and treatment 146(1):211-220.

32. Bruna A, et al. (2016) A Biobank of Breast Cancer Explants with Preserved Intra-tumor Heterogeneity to Screen Anticancer Compounds. Cell 167(1):260-274 .e222.

33. Mjelle R, et al. (2015) Cell cycle regulation of human DNA repair and chromatin remodeling genes. DNA Repair (Amst) 30:53-67.

34. Nalepa G & Clapp D W (2018) Fanconi anaemia and cancer: an intricate relationship. Nature reviews. Cancer 18(3):168-185.

35. Murray J M & Can A M (2018) Integrating DNA damage repair with the cell cycle. Curr Opin Cell Biol 52:120-125.

36. Hurtado A, Holmes K A, Ross-Innes C S, Schmidt D, & Carroll J S (2011) FOXA1 is a key determinant of estrogen receptor function and endocrine response. Nat Genet 43(1):27-33.

37. Joseph R, et al. (2010) Integrative model of genomic factors for determining binding site selection by estrogen receptor-alpha. Mol Syst Biol 6:456.

38. Abdel-Fatah T M, et al. (2014) Is there a role for base excision repair in estrogen/estrogen receptor-driven breast cancers? Antioxidants & redox signaling 21(16):2262-2268.

39. Kwei K A, Kung Y, Salari K, Holcomb I N, & Pollack J R (2010) Genomic instability in breast cancer: pathogenesis and clinical implications. Molecular oncology 4(3):255-266.

40. Liberzon A, et al. (2015) The Molecular Signatures Database (MSigDB) hallmark gene set collection. Cell systems 1(6):417-425.

41. Liberzon A, et al. (2011) Molecular signatures database (MSigDB) 3.0. Bioinformatics (Oxford, England) 27(12):1739-1740.

42. Cline M S, et al. (Integration of biological networks and gene expression data using Cytoscape

Cytoscape: a software environment for integrated models of biomolecular interaction networks. (1750-2799 (Electronic)).

43. Parker J S, et al. (2009) Supervised risk predictor of breast cancer based on intrinsic subtypes. Journal of clinical oncology: official journal of the American Society of Clinical Oncology 27(8):1160-1167.

44. Zehir A, et al. (2017) Mutational landscape of metastatic cancer revealed from prospective clinical sequencing of 10,000 patients. Nat Med 23(6):703-713.

45. Hovelson D H, et al. (2015) Development and validation of a scalable next-generation sequencing system for assessing relevant somatic variants in solid tumors. Neoplasia (New York, N.Y.) 17(4):385-399.

46. Rhodes D R, et al. (2007) Oncomine 3.0: genes, pathways, and networks in a collection of 18,000 cancer gene expression profiles. Neoplasia (New York, N.Y.) 9(2):166-180.

47. Rhodes D R, et al. (2004) ONCOMINE: a cancer microarray database and integrated data-mining platform. Neoplasia (New York, N.Y.) 6(1):1-6.

48. Benjamin Y & Yekutieli D (2001) The Control of the False Discovery Rate in Multiple Testing under Dependency. The Annals of Statistics 29(4):1165-1188.

49. Wang J, Vasaikar S, Shi Z, Greer M, & Zhang B (2017) WebGestalt 2017: a more comprehensive, powerful, flexible and interactive gene set enrichment analysis toolkit. Nucleic acids research.

50. Haricharan S & Brown P (2015) TLR4 has a TP53-dependent dual role in regulating breast cancer cell growth. Proceedings of the National Academy of Sciences of the United States of America 112(25):E3216-3225.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of determining efficacy of one or more cyclin D-dependent kinase (CDK) 4/6 inhibitors as therapy for an individual with ER+ cancer, comprising the step of determining in a sample from the individual the level of expression and/or presence of mutation in Centrin 2 (CETN2), excision repair cross -complementation group 1 (ERCC1), Nei Like DNA Glycosylase 2 (NEIL2), mutL homolog 1 (MLH1), and PMS1 homolog 2, mismatch repair system component (PMS2).

2. The method of claim 1, wherein when the expression level of CETN2, ERCC1, NEIL2, MLH1, and PMS2 in the sample from the individual is reduced compared to a standard or compared to normal levels and/or when there is a mutation in CETN2, ERCC1, NEIL2, MLH1, and PMS2, the individual is provided one or more CDK-4/6 inhibitors.

3. The method of claim 1, further comprising the step of determining the level of expression and/or presence of mutation of PMS 1 homolog 1, mismatch repair system component (PMS 1) in the sample and/or determining copy number loss or mutation of Protein Kinase, DNA-Activated, Catalytic Polypeptide (PRKDC).

4. The method of claim 1, wherein the one or more CDK-4/6 inhibitors are abemaciclib, palbociclib, ribociclib, or a combination thereof.

5. The method of claim 1, wherein the ER+ cancer is ER+ breast, bladder, colorectal, lung, ovarian, prostate, stomach or hepatic cancer.

6. The method of claim 1, wherein the individual is provided hormone therapy, surgery, chemotherapy, or radiation for the cancer.

7. The method of claim 1, wherein the sample is biopsy, urine, feces, polyp, cerebrospinal fluid, blood, semen, nipple aspirate, or a combination thereof.

8. A method of treating an individual for ER+ cancer, comprising the step of providing to an individual with reduced expression levels of CETN2, ERCC1, NEIL2, MLH1, and PMS2 compared to a standard or compared to normal levels, and/or has one or more mutations in CETN2, ERCC1, NEIL2, MLH1, and PMS2, an effective amount of one or more CDK-4/6 inhibitors.

9. A method of treating an individual for ER+ cancer, comprising the steps of:
(a) identifying a subject susceptible to treatment of ER+ cancer by:
(1) measuring from a sample from the individual the level of expression of CETN2, ERCC1, NEIL2, MLH1, and PMS2 compared to a standard or compared to normal levels; and/or
(2) assaying from a sample from the individual for one or more mutations in the gene, mRNA, or protein of CETN2, ERCC1, NEIL2, MLH1, and PMS2; and
(b) administering an effective amount of one or more CDK-4/6 inhibitors to the individual that is susceptible to treatment of ER+ cancer by having reduced expression levels of CETN2, ERCC1, NEIL2, MLH1, and PMS2 and/or by having one or more mutations in each of CETN2, ERCC1, NEIL2, MLH1, and PMS2.

10. The method of claim 8, wherein the individual is provided one or more therapies for the ER+ cancer other than the one or more CDK-4/6 inhibitors.

11. The method of claim 10, wherein the one or more other therapies comprises hormone therapy, surgery, radiation, or a combination thereof.

12. The method of claim 11, wherein the hormone therapy comprises one or more selective estrogen-receptor response modulators (SERMs); one or more Aromatase inhibitors; one or more Estrogen-receptor downregulators (ERDs); one or more Luteinizing hormone-releasing hormone agents (LHRHs); or a combination thereof.

13. The method of claim 8, further comprising the step of measuring the level of expression and/or assaying for mutation in PMS1 and/or determining copy number loss or mutation of PRKDC.

14. The method of claim 8, wherein the one or more CDK-4/6 inhibitors are abemaciclib, palbociclib, ribociclib, or a combination thereof.

15. The method of claim 8, wherein the ER+ cancer is breast, bladder, colorectal, lung, ovarian, prostate, stomach or hepatic cancer.

16. The method of claim 8, wherein the sample comprises biopsy, urine, feces, polyp, cerebrospinal fluid, blood, semen, nipple aspirate, or a combination thereof.

17. A method for treating an individual with ER+ cancer, the method comprising treating the patient for ER+ cancer after the expression level of one or more of CETN2, ERCC1, NEIL2, MLH1, and PMS2 has been determined in a sample from the individual and/or when the nucleotide and/or amino acid sequence of CETN2, ERCC1, NEIL2, MLH1, and PMS2 has been determined in a sample from the patient.

18. A method for evaluating a ER+ cancer patient comprising measuring the level of expression of one or more of CETN2, ERCC1, NEIL2, MLH1, and PMS2 in a sample from the patient and/or assaying for mutation in one or more of CETN2, ERCC 1 , NEIL2, MLH 1 , and PMS2 in a sample from the patient.

19. A method of prognosing and/or diagnosing a patient comprising
a) measuring the level of expression of one or more of CETN2, ERCC1, NEIL2, MLH1, and PMS2 in a sample from the patient;
b) comparing the level of expression to a control sample(s) or control level(s) of expression; and,
c) prognosing or diagnosing the patient based on the levels of measured expression.

20. The method of claim 1, wherein the individual or patient has been determined to have a familial history of ER+ cancer or wherein the individual or patient has been determined not to have a familial history of ER+ cancer.

21. (canceled)

22. The method of claim 1, wherein the individual or patient has or has not had a prior screening procedure for ER+ cancer.

23. (canceled)

24. The method of claim 1, wherein the patient has not been diagnosed with ER+ cancer.

25. The method of claim 1, wherein the expression level of no other ER+ cancer biomarker in the biological sample was determined.
